(19) United States
(12) Patent Application Publication
(10) Pub. No.: US 2010/0099745 A1
(43) Pub. Date: Apr. 22, 2010

(54) ENHANCING DISEASE RESISTANCE AGAINST RNA VIRAL INFECTIONS WITH INTRACYTOPLASMIC PATHOGEN SENSORS

(76) Inventors: Suryaprakash Sambhara, Atlanta, GA (US); Zha Guo, Tucker, GA (US)

Correspondence Address: KLARQUIST SPARKMAN, LLP, 121 S.W. SALMON STREET, SUITE 1600, PORTLAND, OR, 97204, US

(21) Appl. No.: 12/445,010

(22) PCT Filed: Oct. 16, 2007
(86) PCT No.: PCT/US2007/081545

§ 371 Date: Apr. 9, 2009

Related U.S. Application Data
(60) Provisional application No. 60/852,727, filed on Oct. 18, 2006.

(51) Int. Cl. A61K 31/7088; C12N 15/74; A61P 31/12

(52) U.S. Cl. 514/44 R; 435/320.1

(57) ABSTRACT

The present disclosure provides compositions and methods for enhancing resistance to viral infections. The compositions include adenovirus vectors containing nucleic acid molecules encoding CARD domains from RIG-I and MDA5, recombinant adenoviruses and immunogenic compositions comprising such recombinant adenovirus vectors and adenoviruses. Methods for enhancing resistance to viral infections involving administering such adenovirus vectors or recombinant adenovirus are also provided.

### CROSS REFERENCE TO RELATED APPLICATION

This application claims the benefit of U.S. Provisional Application No. 60/852,727, filed Oct. 18, 2006, which is incorporated by reference herein in its entirety.

### FIELD

This application relates to the field of resistance to viral infection. More specifically, this application concerns recombinant vectors for the production of polypeptides that enhance viral resistance and enhancing the immunogenicity of the vaccines.

### BACKGROUND

The innate immune system is the host's first line of defense against a variety of pathogens. One of the major mechanisms for rapid initiation of host innate immune responses is to recognize conserved motifs or pathogen-associated molecule patterns (PAMPs) unique to pathogens by pattern recognition receptors, such as Toll-like receptors (TLRs) (Kaisho and Akira, J. Allergy Clin. Immunol. 117, 979-987, 2006). Upon recognition of PAMPs, pattern recognition receptors activate signaling pathways that lead to secretion of proinflammatory cytokines, such as type I interferon (IFN-I) that are essential in antiviral immunity. IFN-I can be induced by binding of a variety of pathogen constituents or by products of infection, such as intracellular double-stranded RNA (dsRNA), extracellular dsRNA, lipopolysaccharide, single-stranded RNA (ssRNA), and unmethylated CpG DNA (Kaisho and Akira, J Allergy Clin Immunol. 117, 979-987, 2006; Yoneyama et al., Nat. Immunol. 5, 730-737, 2004).

Several human viruses, including hepatitis C virus (HCV, Li et al., Proc. Natl. Acad. Sci. U.S.A. 102, 2992-2997, 2005), vaccinia virus (Smith et al., J. Biol. Chem. 276, 8951-8957, 2001), Ebola virus (Basler et al., J. Virol. 77, 7945-7956, 2003), and influenza virus (Talon et al., J. Virol. 74, 7989-7996, 2000), have evolved strategies to target and inhibit distinct steps in the early signaling events that lead to IFN-I induction, indicating the importance of IFN-I in the host's antiviral response. For example, the viral protease NS3/4A encoded by HCV has recently been shown to block the activation of interferon regulatory factor 3 (IRF-3) by inactivating the adaptor proteins TRIF and IPS-1 to prevent IFN-I production (Li et al., Proc. Natl. Acad. Sci. U.S.A. 102, 2992-2997, 2005; Foy et al., Proc. Natl. Acad. Sci. U.S.A. 102, 2986-2991, 2005; Meylan et al., Nature 437, 1167-1172, 2005). It also has been suggested that sequestering of viral dsRNA by nonstructural protein 1 (NS1) of influenza A virus (IAV) during virus replication prevents access of host dsRNA sensors (Talon et al., J. Virol. 74, 7989-7996, 2000), limiting the induction of IFN-I. The role of NS1 of IAV as an IFN antagonist is evidenced by the hyper-induction of IFN-I in response to IAV lacking the NS1 gene (delNS1 virus) as compared to wild type virus infection (Talon et al., J Virol 74, 7989-7996, 2000; Donelan et al. J. Virol. 77, 13257-13266, 2003; Wang et al., J. Virol. 74, 11566-11573, 2000). Additionally, ectopic expression of NS1 inhibits activation of IRF-3 (Talon et al., J. Virol. 74, 7989-7996, 2000).

The need exists for compositions that confer protective immunity against viral infection, by circumventing the ability of the viruses to inhibit IFN-I induction. The present disclosure addresses this need, and provides novel compositions and methods useful for stimulating innate immunity, thereby inhibiting viral infection as well as enhancing immune responses to vaccines.

### SUMMARY

Methods of inhibiting viral infection (such as a viral infection from an RNA virus for example a ssRNA virus such as influenza virus, or a dsRNA virus) in a subject are disclosed. These methods include selecting a subject in which the viral infection is to be inhibited and administering an effective amount of a recombinant adenovirus vector containing a nucleic acid sequence encoding at least one caspase recruitment domain (CARD) from MDA5 or RIG-I. The methods can also include administering a viral vaccine to the subject. In some examples, the vaccine is an influenza vaccine, such as a vaccine against one or more avian or pandemic strains of influenza, for example influenza strains H5N1, H7N7, H9N2, or a combination thereof. Optionally, Flt3 ligand can be administered to a subject as an adjuvant. In particularly effective examples the adenoviral vector does not contain a nucleotide sequence encoding a helicase domain, so that the CARD domains are constitutively active and are able to stimulate an immune response for example by induction of interferon such as interferon type 1.

Also disclosed are adenoviral vectors and adenoviruses that contain nucleic acids encoding CARDs, such as CARDs from MDA5 and/or RIG-I. In particularly effective examples the adenoviral vector does not contain a nucleotide sequence encoding a helicase domain, so that the CARD domains are constitutively active and are able to stimulate an immune response for example by induction of interferon such as interferon type 1. In some examples, the disclosed adenovirus vectors contain at least one additional heterologous nucleic acid sequence that encodes a polypeptide, such as at least one viral antigen polypeptide and/or a Flt3 ligand polypeptide. Pharmaceutical compositions containing the recombinant adenovirus vectors and adenoviruses are also disclosed.

The foregoing and other objects, features, and advantages of the invention will become more apparent from the following detailed description, which proceeds with reference to the accompanying figures.

### BRIEF DESCRIPTION OF THE DRAWINGS

FIGS. 1A-1H are a set of bar graphs and a digital image of an immunoblot, demonstrating that RIG-I is involved in the induction of type I interferon (IFN-I) against influenza A virus (IAV) infection. A549 cells were transfected with siRNA targeting RIG-I (siRIG-I) or control siRNA targeting luciferase gene (siLuc). After a 24 hour incubation, transfected cells were infected with influenza virus A/Panama/2007/99 and incubated for 16 hours. Total RNA was isolated, and real-time RT-PCR was performed to analyze IFNβ (FIG. 1A), ISG15 (FIG. 1B), MxA (FIG. 1C), TNF-α (FIG. 1D), and RIG-I (FIG. 1G) expression. For reporter assay and protein analysis, A549 cells were transiently co-transfected with siRNA and reporter plasmids as indicated, followed by infection with IAV PR8. Cell lysates were collected and analyzed by CAT ELISA (FIG. 1E and FIG. 1F), or by western blot analysis using antibodies against RIG-I or β-actin (FIG. 1H). The average of three independent trials is shown with S.D.

FIGS. 2A and 2B are a digital image of an immunoblot and a set of bar graphs, demonstrating that MDA5 is a component for the induction of type I interferon against influenza A virus infection. A549 cells were transfected with siRNA targeting MDA5 (siMDA5), RIG-I (siRIG-I), or control siRNA targeting luciferase gene (siLuc). After a 24 hour incubation, transfected cells were infected with IAV PR8 and incubated for 16 hours. FIG. 2A is a digital image of an immunoblot. Cell lysates were collected and analyzed by western blot analysis using antibodies against MDA5 or β-actin. FIG. 2B is a set of bar graphs showing the relative levels of IFNβ, ISG15, MxA, and TNF-α in treated cells. Total RNA was isolated, and real-time RT-PCR was performed to analyze the expression of IFNβ, ISG15, MxA, and TNF-α. The relative levels of mRNA expression were plotted as fold of increase with IAV-infected mock controls being set as 1-fold.

FIGS. 3A and 3B are a bar graph and a digital image of an immunoblot, demonstrating that the C-terminal helicase domain of RIG-I functions as a dominant negative inhibitor for IFNβ production induced by IAV infection. 293T cells were transiently transfected with IFNβ promoter reporter plasmid DNA together with various amounts of control vector pEF-BOS, or vectors that express FLAG-tagged C-terminal domain or full-length of human RIG-I. After a 24 hour incubation, cells were infected with IAV PR8 and incubated for another 24 hours. Cell lysates were collected and a CAT ELISA was performed. The average of three independent trials is shown with S.D. in FIG. 3A. Samples tested by CAT ELISA shown in FIG. 3A were also analyzed by western blot using antibodies against FLAG-tag or β-actin as shown in the digital image of the immunoblot in FIG. 3B.

FIGS. 4A-4G are a set of bar graphs and digital images of immunoblots, demonstrating that NS1 from influenza A virus antagonizes production of IFNβ induced by RIG-I. FIG. 4A, IFNβ-CAT reporter and FLAG-tagged RIG-I expression vectors were transiently transfected with increased amounts of the myc-tagged NS1 expression vector into A549 cells. Cell lysates were collected 24 hours post transfection and analyzed by CAT ELISA. FIG. 4B, A549 cells were transfected with vectors that express FLAG-tagged RIG-I or myc-tagged NS1, or their corresponding control vectors pEF-BOS or pCAGGS as indicated. After 24 hours of incubation, cells were collected and total RNA was isolated, followed by real time RT-PCR analysis for the expression of IFNβ, ISG15, MxA and TNF-α. FIG. 4C is a digital image that shows a western blot was performed to confirm the ectopic expression of RIG-I and NS1 using antibodies against FLAG-tag or myc-tag. FIG. 4D-4F, 293T cells were transiently transfected with indicated promoter reporter plasmids together with vectors that express FLAG-tagged RIG-I or myc-tagged NS1. After 24 hours of incubation, cells were transfected with poly (I:C) and incubated for another 24 hours. Cell lysates were collected and analyzed by CAT ELISA to determine activities of the IFNβ promoter (FIG. 4D) and IRF-3-responsive promoter (FIG. 4E), or analyzed by western blot analysis using antibodies against FLAG-tag or myc-tag (FIG. 4F). FIG. 4G, IFNβ-CAT reporter plasmids and vectors that expressed RIG-I, IPS1, TRIF, or IKKε were co-transfected with or without the myc-tagged NS1 expression vectors into A549 cells. Cell lysates were collected 24 hours post transfection and analyzed by CAT ELISA. The relative levels of CAT expression were plotted as fold of increase with samples transfected with pCAGGS and adaptor expression vectors being set as 1-fold. The average of three independent trials is shown with S.D.

FIGS. 5A and 5B are a bar graph and a digital image of an immunoblot, demonstrating that NS1 from IAV antagonizes RIG-I signaling through its N-terminal domain. A549 cells were transiently transfected with IFNβ-CAT reporter plasmids together with vectors that expressed FLAG-tagged RIG-I domains or myc-tagged NS1 domains. After 24 hours of incubation, cell lysates were collected and analyzed by CAT ELISA (FIG. 5A), or analyzed by western blot analysis using antibodies against FLAG-tag or myc-tag (FIG. 5B).

FIGS. 6A and 6B are a set of bar graphs demonstrating that RIG-I inhibits replication of highly pathogenic avian influenza A virus. A549 cells were transiently transfected with control vector pEF-BOS or the vector that expresses full-length RIG-I. After 24 hours of incubation, cells were infected with IAV PR8 (H1N1, FIG. 6A) or highly pathogenic avian IAV A/Vietnam/1203/2004 (H5N1, FIG. 6B) at various MOIs and incubated for another 24 hours. Culture supernatants were collected and viral titers were determined by plaque assay on MDCK cells. The average of three independent trials is shown with S.D.

FIGS. 7A and 7B are a set of bar graphs demonstrating the effect of NS1 on the production of interferon β. FIG. 7A demonstrates the production of interferon β in the presence of RIG-I is reduced in the presence of NS1. FIG. 7B shows that NS1 reduces the transcription of LacZ in the presence of I:C double stranded nucleic acids.

FIGS. 8A, 8B and 8C are schematic representations of adenoviral vector constructs containing expressing green fluorescent protein (GFP) and FLAG tagged C-terminal domain of RIG-I (AD-VEC-FLAG-C-TER-RIG-I (expressing from amino acid 218 through the stop codon of RIG-I with an N-terminal FLAG tag)), FLAG tagged N-terminal domain or RIG-I (AD-VEC-FLAG-N-TER-RIG-I (expressing the first 228 amino acids of RIG-I with an N-terminal FLAG tag)), and FLAG tagged full length RIG-I (AD-VEC-FLAG-FULL-RIG-I (expressing full length RIG-I protein with an N-terminal FLAG tag)), respectively.

FIG. 9 are a set of digital images of a fluorescent microscope images of A549 cells infected with the indicated adenoviruses co-expressing RIG-I constructs and GFP.

FIG. 10 are a set of digital images of Western blots of A549 cells infected with the indicated GFP expressing adenoviral vector constructs, showing that cells infected with an adenoviral vector construct containing both GFP and full length RIG-I express both GFP and RIG1 (lane 3).

### SEQUENCE LISTING

The nucleic and amino acid sequences listed in the accompanying sequence listing are shown using standard letter abbreviations for nucleotide bases, and one letter code for amino acids, as defined in 37 C.F.R.1.822. Only one strand of each nucleic acid sequence is shown, but the complementary strand is understood as included by any reference to the displayed strand.

SEQ ID NO:1 is an exemplary amino acid sequence of RIG-I.

SEQ ID NO:2 is an exemplary nucleic acid sequence of RIG-I.

SEQ ID NO:3 is an exemplary amino acid sequence of MDA5.

SEQ ID NO:4 is an exemplary nucleic acid sequence of MDA5.

SEQ ID NO:5 is an exemplary amino acid sequence of an HA epitope.

SEQ ID NO:6 is an exemplary amino acid sequence of an NP epitope.

### DETAILED DESCRIPTION OF SEVERAL EMBODIMENTS

### I. Abbreviations

APC: antigen presenting cells

CARD: caspase recruitment domain

DC: Dendritic cell

dsRNA: double-stranded RNA

HA: hemagglutinin

HCV: hepatitis C virus

IAV: influenza A virus

IFN-β: interferon-β

IFN-I: type I interferon

MDA5: melanoma differentiation associated protein-5

NA: neuraminidase

NS1: nonstructural protein 1

PAMP: pathogen-associated molecular patterns

ssRNA: single-stranded RNA

TLR: toll-like receptor

### II. Terms

Unless otherwise noted, technical terms are used according to conventional usage. Definitions of common terms in molecular biology can be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.” In case of conflict, the present specification, including explanations of terms, will control. In addition, all the materials, methods, and examples are illustrative and not intended to be limiting.

To facilitate review of the various embodiments of the disclosure, the following explanations of specific terms are provided:

Animal: Living multi-cellular vertebrate organisms, a category that includes, for example, mammals and birds. The term mammal includes both human and non-human mammals. Similarly, the term “subject” includes both human and non-human subjects, including birds and non-human mammals, such as non-human primates.

Antibody: A polypeptide substantially encoded by an immunoglobulin gene or immunoglobulin genes, or fragments thereof, which specifically binds and recognizes an analyte (antigen), such as a viral antigen. Immunoglobulin genes include the kappa, lambda, alpha, gamma, delta, epsilon and mu constant region genes, as well as the myriad immunoglobulin variable region genes.

Antibodies exist, for example as intact immunoglobulins and as a number of well characterized fragments produced by digestion with various peptidases. For instance, Fabs, Fvs, and single-chain Fvs (scFvs) that bind to a viral antigen are specific binding agents. This includes intact immunoglobulins and the variants and portions of them well known in the art, such as Fab′ fragments, F(ab)′₂ fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes genetically engineered forms such as chimeric antibodies (such as humanized murine antibodies), heteroconjugate antibodies such as bispecific antibodies). See also, Pierce Catalog and Handbook, 1994-1995 (Pierce Chemical Co., Rockford, Ill.); Kuby, J., Immunology, 3^{rd} Ed., W.H. Freeman & Co., New York, 1997.

Antigen: A compound, composition, or substance that can stimulate the production of antibodies or a T cell response in an animal, including compositions that are injected, absorbed or otherwise introduced into an animal. The term “antigen” includes all related antigenic epitopes. An “antigenic polypeptide” is a polypeptide to which an immune response, such as a T cell response or an antibody response, can be stimulated. “Epitope” or “antigenic determinant” refers to a site on an antigen to which B and/or T cells respond. In one embodiment, T cells respond to the epitope when the epitope is presented in conjunction with an MHC molecule. Epitopes can be formed both from contiguous amino acids or noncontiguous amino acids juxtaposed by tertiary folding of an antigenic polypeptide. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, or about 8-10 amino acids in a unique spatial conformation. Methods of determining spatial conformation of epitopes include, for example, x-ray crystallography and multi-dimensional nuclear magnetic resonance spectroscopy.

In some examples an antigen is a viral antigen. For example an antigen can be a polypeptide expressed on the surface of a virus, such as a viral envelope protein. In another example an antigen is an internal viral protein. Examples of antigens include, antigens selected from animal and human viral pathogens, such as influenza, RSV, HIV, Rotavirus, New Castle Disease Virus, Marek Disease Virus, Metapneumovirus, Parainfluenza viruses, Coronaviruses (including for example, SARS-CoV, HCoV-HKU1, HCoV-NL63 and TGEV), Hepatitis C virus, Flaviviruses (such as Dengue virus, Japanese Encephlitis virus, Kunjin virus, Yellow fever virus and West Nile virus), Filoviruses (such as Ebola virus and Marburg Virus), Caliciviruses (including Norovirus and Sapovirus), Human Papilloma Virus, Epstein Barr Virus, Cytomegalovirus, Varicella Zoster virus, and Herpes Simplex Virus among others. Non-limiting examples of antigens include: influenza antigen (such as hemagglutinin (HA), neuraminidase (NA) antigen, or an influenza internal protein, such as a PB1, PB2, PA, M1, M2, NP, NS1 or NS2 protein); RSV (Type A & B) F and G proteins; HIV p24, pol, gp41 and gp120; Rotavirus VP8 epitopes; New Castle Disease Virus F and HN proteins; Marek Disease Virus Glycoproteins: gB, gC, gD, gE, gH, gI, and gL; Metapneumovirus F and G proteins; Parainfluenza viruses F and HN proteins; Coronavirus (e.g. SARS-CoV, HCoV-HKU1, HCoV-NL63, TGEV) S, M and N proteins; Hepatitis C virus E1, E2 and core proteins; Dengue virus E1, E2 and core proteins; Japanese encephalitis virus E1, E2 and core proteins; Kunjin virus E1, E2 and core proteins; West Nile virus E1, E2 and core proteins; Yellow Fever virus E1, E2 and core proteins; Ebola virus and Marburg Virus structural glycoprotein; Norovirus and Sapovirus major capsid proteins; Human Papilloma Virus L1 protein; Epstein Barr Virus gp220/350 and EBNA-3A peptide; Cytomegalovirus gB glycoprotein, gH glycoprotein, pp65, IE1 (exon 4) and pp150; Varicella Zoster virus IE62 peptide and glycoprotein E epitopes; Herpes Simplex Virus Glycoprotein D epitopes, among many others. In some examples the antigen is a tumor antigen.

A variant of an antigen can be a naturally occurring variant or an engineered variant. As used herein, the term “variant” refers to a protein (for example, an antigen) with one or more amino acid alterations, such as deletions, additions or substitutions, relative to a reference protein or with respect to another variant.

Caspase Recruitment Domain or CARD: “CARD” is an interaction motif found in a wide array of proteins. Typically, CARDs are about 80 to 110 amino acids in length. CARDs are a subclass of protein motif known as the death fold, which features an arrangement of six to seven antiparallel alpha helices with a hydrophobic core and an outer face composed of charged residues. CARDs mediate the formation of larger protein complexes via direct interactions between individual CARDs. CARD/CARD interactions are believed to be mediated primarily by electrostatic interactions between complementary charged surfaces with a binding specificity achieved by particular charge patterns between CARD binding partners. For example a CARD with a basic surface interacts with a CARD with a complementary acidic surface.

A subset of CARD containing proteins, RIG-I and MDA5, participate in recognition of intracellular RNA, such as double-stranded RNA. As used herein a RIG-I CARD refers to a CARD that is at least 95% identical to residues 1 to 87 of the amino acid sequence set forth as SEQ ID NO:1 or is at least 95% identical to residues 92-172 of the amino acid sequence set forth as SEQ ID NO:1 and is capable of forming a dimer with its binding partner. As used herein an MDA5 CARD refers to a CARD that is at least 95% identical to residues 7 to 97 of the amino acid sequence set forth as SEQ ID NO:3 or is at least 95% identical to residues 110 to 190 of the amino acid sequence set forth as SEQ ID NO:3 and is capable of forming a dimer with its binding partner.

Dendritic cell (DC): Dendritic cells are the principal antigen presenting cells (APCs) involved in primary immune responses. Their major function is to obtain antigen in tissues, migrate to lymphoid organs, and present the antigen in order to activate T-cells.

When an appropriate maturational cue is received, DCs are signaled to undergo rapid morphological and physiological changes that facilitate the initiation and development of immune responses. Among these are the up-regulation of molecules involved in antigen presentation; production of pro-inflammatory cytokines, including IL-12, key to the generation of Th1 responses; and secretion of chemokines that help to drive differentiation, expansion, and migration of surrounding naive Th cells. Collectively, these up-regulated molecules facilitate the ability of DCs to coordinate the activation and effector function of other surrounding lymphocytes that ultimately provide protection for the host. Although the process of DCs maturation is commonly associated with events that lead to the generation of adaptive immunity, many stimuli derived from the innate branch of the immune system are also capable of activating DCs to initiate this process. In this manner, DCs provide a link between the two branches of the immune response, in which their initial activation during the innate response can influence both the nature and magnitude of the ensuing adaptive response. A dendritic cell precursor is a cell that matures into an antigen presenting dendritic cell.

Degenerate variant and conservative variant: A polynucleotide encoding a polypeptide or an antibody that includes a sequence that is degenerate as a result of the genetic code. For example, a polynucleotide encoding a RIG-I polypeptide includes a sequence that is degenerate as a result of the genetic code. There are 20 natural amino acids, most of which are specified by more than one codon. Therefore, all degenerate nucleotide sequences are included as long as the amino acid sequence of the RIG-I polypeptide encoded by the nucleotide sequence is unchanged. Because of the degeneracy of the genetic code, a large number of functionally identical nucleic acids encode any given polypeptide. For instance, the codons CGU, CGC, CGA, CGG, AGA, and AGG all encode the amino acid arginine. Thus, at every position where an arginine is specified within a protein encoding sequence, the codon can be altered to any of the corresponding codons described without altering the encoded protein. Such nucleic acid variations are “silent variations,” which are one species of conservative variations. Each nucleic acid sequence herein that encodes a polypeptide also describes every possible silent variation. One of skill will recognize that each codon in a nucleic acid (except AUG, which is ordinarily the only codon for methionine) can be modified to yield a functionally identical molecule by standard techniques. Accordingly, each “silent variation” of a nucleic acid which encodes a polypeptide is implicit in each described sequence.

Furthermore, one of ordinary skill will recognize that individual substitutions, deletions or additions which alter, add or delete a single amino acid or a small percentage of amino acids (for instance less than 5%, in some embodiments less than 1%) in an encoded sequence are conservative variations where the alterations result in the substitution of an amino acid with a chemically similar amino acid.

Conservative amino acid substitutions providing functionally similar amino acids are well known in the art. The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

Not all residue positions within a protein will tolerate an otherwise “conservative” substitution. For instance, if an amino acid residue is essential for a function of the protein, even an otherwise conservative substitution may disrupt that activity.

Enhancing Vaccine Effectiveness: Refers to the ability of an agent (for example an adenoviral vector encoding a CARD from RIG-I of MDA5) to increase the ability of a vaccine to induce a protective immune response in a subject relative to the vaccine alone.

Expression: Translation of a nucleic acid into a protein. Proteins may be expressed and remain intracellular, become a component of the cell surface membrane, or be secreted into the extracellular matrix or medium.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see for example, Bitter et al., Methods in Enzymology 153:516-544, 1987). For example, when cloning in bacterial systems, inducible promoters such as pL of bacteriophage lambda, plac, ptrp, ptac (ptrp-lac hybrid promoter) and the like may be used. In one embodiment, when cloning in mammalian cell systems, promoters derived from the genome of mammalian cells (such as metallothionein promoter) or from mammalian viruses (such as the retrovirus long terminal repeat; the adenovirus late promoter; the vaccinia virus 7.5K promoter) can be used. Promoters produced by recombinant DNA or synthetic techniques may also be used to provide for transcription of the nucleic acid sequences.

A polynucleotide can be inserted into an expression vector that contains a promoter sequence which facilitates the efficient transcription of the inserted genetic sequence of the host. The expression vector typically contains an origin of replication, a promoter, as well as specific nucleic acid sequences that allow phenotypic selection of the transformed cells.

Flt3 Ligand: The Flt3 (fms-like tyrosine kinase 3)/Flk2 (fetal liver kinase 2) ligand is a hematopoietic cytokine that binds to Flt3 tyrosine kinase receptor. Human Flt3 ligand is a type I transmembrane glycoprotein that can be cleaved to generate a soluble form that is also biologically active. As used herein Flt3 ligand refers to both the cell surface glycoprotein and soluble forms of the protein. Flt3 ligand stimulation of Flt3 receptor tyrosine kinase expands early hematopoietic progenitor and dendritic cells (DCs). Exemplary amino acid sequences of Flt3 ligand are available (see, for example, GENBANK® Accession No. AAA19825).

Human adenovirus vectors: An adenovirus vector of human origin. A “non-human adenovirus vector” is an adenoviral vector of non-human origin.

Helicase domain: A protein domain capable of binding to double stranded nucleic acids (such as dsRNA or dsDNA) and unwinding double stranded nucleic acids in a ATP dependent manner.

Immune response: A response of a cell of the immune system, such as a B cell, T cell, Natural Killer cell, or monocyte, to a stimulus. In one embodiment, the response is specific for a particular antigen (an “antigen-specific response”). In one embodiment, an immune response is a T cell response, such as a CD4+ response or a CD8+ response. In another embodiment, the response is a B cell response, and results in the production of specific antibodies.

Immunogenic composition: A composition comprising an immunogenic peptide that induces a measurable cytotoxic T cell (CTL) response against virus expressing the immunogenic peptide, or induces a measurable B cell response (such as production of antibodies) against the immunogenic peptide. In one example an “immunogenic composition” is a composition comprising viral antigen that induces a measurable CTL response against virus expressing the viral antigen, or induces a measurable B cell response (such as production of antibodies) against a the viral antigen. It further refers to isolated nucleic acids encoding an immunogenic peptide, such as a nucleic acid that can be used to express the viral antigen (and thus be used to elicit an immune response against this polypeptide).

For in vitro use, an immunogenic composition may consist of the isolated protein, peptide epitope, or nucleic acid encoding the protein, or peptide epitope. Any particular peptide, such as a viral antigen, or nucleic acid encoding the polypeptide, can be readily tested for its ability to induce a CTL or B cell response by art-recognized assays. Immunogenic compositions can include adjuvants, which are well known to one of skill in the art. In some examples, an immunogenic composition includes a polypeptide or a nucleic acid molecule encoding a polypeptide of a viral antigen, such as an antigen from an RNA virus such as a dsRNA virus or a ssRNA virus such as an influenza virus.

Immunotherapy: A method of evoking an immune response against a virus based on their production of target antigens or induction of an antiviral state. Immunotherapy based on cell-mediated immune responses involves generating a cell-mediated response to cells that produce particular antigenic determinants, while immunotherapy based on humoral immune responses involves generating specific antibodies to virus that produce particular antigenic determinants. Induction of anti-viral state involves stimulating the target tissue to secrete anti-viral cytokines such as type 1 interferons.

Inhibit: To reduce by a measurable degree.

Isolated: An “isolated” biological component (such as a nucleic acid, peptide or protein) has been substantially separated, produced apart from, or purified away from other biological components in the cell of the organism in which the component naturally occurs, such as, other chromosomal and extrachromosomal DNA and RNA, and proteins. Nucleic acids, peptides and proteins which have been “isolated” thus include nucleic acids and proteins purified by standard purification methods. The term also embraces nucleic acids, peptides, and proteins prepared by recombinant expression in a host cell as well as chemically synthesized nucleic acids.

MDA5: melanoma differentiation associated protein-5 (MDA5) is an intracellular DExD/H box-RNA helicase with a C-terminal helicase domain that binds double-stranded RNA (dsRNA) and two N-terminal caspase recruitment domain (CARD) domains. MDA5 senses intracellular viral double stranded RNA and stimulates the coordinated activation of multiple transcription factors, including NF-κB, IRF-3. The transcription factors act together to regulate the expression of type-1 interferons, such as interferon-β (IFN-β). Thus MDA5 promotes the response to viral infection. MDA5 recognizes the dsRNA of the positive-sense ssRNA virus, encephalomyocarditis virus (which is a picornavirus) among others.

Nucleic acid: A polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs, such as, for example and without limitation, phosphorothioates, phosphoramidates, methyl phosphonates, chiral-methyl phosphonates, 2-O-methyl ribonucleotides, peptide-nucleic acids (PNAs), and the like. Such polynucleotides can be synthesized, for example, using an automated DNA synthesizer. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

“Nucleotide” includes, but is not limited to, a monomer that includes a base linked to a sugar, such as a pyrimidine, purine or synthetic analogs thereof, or a base linked to an amino acid, as in a peptide nucleic acid (PNA). A nucleotide is one monomer in a polynucleotide. A nucleotide sequence refers to the sequence of bases in a polynucleotide. For example, a RIG-I polynucleotide is a nucleic acid encoding a RIG-I polypeptide.

Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand;” sequences on the DNA strand having the same sequence as an mRNA transcribed from that DNA and which are located 5′ to the 5′-end of the RNA transcript are referred to as “upstream sequences;” sequences on the DNA strand having the same sequence as the RNA and which are 3′ to the 3′ end of the coding RNA transcript are referred to as “downstream sequences.”

“cDNA” refers to a DNA that is complementary or identical to an mRNA, in either single stranded or double stranded form.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (for example, rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Recombinant nucleic acid” refers to a nucleic acid having nucleotide sequences that are not naturally joined together. This includes nucleic acid vectors, such as adenoviral vectors, comprising an amplified or assembled nucleic acid which can be used to transform a suitable host cell. A host cell that comprises the recombinant nucleic acid is referred to as a “recombinant host cell.” The gene is then expressed in the recombinant host cell to produce, such as a “recombinant polypeptide.” A recombinant nucleic acid may serve a non-coding function (such as a promoter, origin of replication, ribosome-binding site, etc.) as well.

A first sequence is an “antisense” with respect to a second sequence if a polynucleotide whose sequence is the first sequence specifically hybridizes with a polynucleotide whose sequence is the second sequence.

For sequence comparison of nucleic acid sequences and amino acids sequences, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters are used. Methods of alignment of sequences for comparison are well known in the art. Optimal alignment of sequences for comparison can be conducted, for example, by the local homology algorithm of Smith & Waterman, Adv. Appl. Math. 2:482, 1981, by the homology alignment algorithm of Needleman & Wunsch, J. Mol. Biol. 48:443, 1970, by the search for similarity method of Pearson & Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see for example, Current Protocols in Molecular Biology (Ausubel et al., eds 1995 supplement)).

Algorithms that are suitable for determining percent sequence identity and sequence similarity are the BLAST and the BLAST 2.0 algorithm, which are described in Altschul et al., J. Mol. Biol. 215:403-410, 1990 and Altschul et al., Nucleic Acids Res. 25:3389-3402, 1977. Software for performing BLAST analyses is publicly available through the National Center for Biotechnology Information (http://www.ncbi.nlm.nih.gov/). The BLASTN program (for nucleotide sequences) uses as defaults a word length (W) of 11, alignments (B) of 50, expectation (E) of 10, M=5, N=−4, and a comparison of both strands. The BLASTP program (for amino acid sequences) uses as defaults a word length (W) of 3, and expectation (E) of 10, and the BLOSUM62 scoring matrix (see Henikoff & Henikoff, Proc. Natl. Acad. Sci. USA 89:10915, 1989).

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Pharmaceutical agent: A chemical compound or composition capable of inducing a desired therapeutic or prophylactic effect when properly administered to a subject or a cell. “Incubating” includes a sufficient amount of time for interaction with a cell. “Contacting” is placement in direct physical association. Includes both in solid and liquid form. Contacting can occur in vitro with isolated cells or in vivo by administering to a subject. “Administrating” to a subject includes topical, parenteral, oral, intravenous, intra-muscular, sub-cutaneous, inhalational, nasal, intra-articular or dermal administration, among others.

An “anti-viral agent” is an agent that specifically inhibits a virus from replicating or infecting cells.

A “therapeutically effective amount” is a quantity of a chemical composition or an anti-viral agent sufficient to achieve a desired effect in a subject being treated. For instance, this can be the amount necessary to inhibit viral replication or to measurably alter outward symptoms of the viral infection, such as a decrease or lack of symptoms associated with a viral infection. In general, this amount will be sufficient to measurably inhibit virus replication or infectivity. When administered to a subject, a dosage will generally be used that will achieve target tissue concentrations that has been shown to achieve in vitro inhibition of viral replication.

Pharmaceutically acceptable carriers: The pharmaceutically acceptable carriers of use are conventional. Remington's Pharmaceutical Sciences, by E. W. Martin, Mack Publishing Co., Easton, Pa., 15th Edition, 1975, describes compositions and formulations suitable for pharmaceutical delivery of the compositions disclosed herein.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Polypeptide: Any chain of amino acids, regardless of length or post-translational modification (such as glycosylation or phosphorylation). “Polypeptide” applies to naturally occurring amino acid polymers and non-naturally occurring amino acid polymers as well as polymers in which one or more amino acid residue is a non-natural amino acid, for example a artificial chemical mimetic of a corresponding naturally occurring amino acid. In one embodiment, the polypeptide is a RIG-I polypeptide, such as a full length RIG-I polypeptide or a portion of RIG-I such as the C-terminal domain or one or more CARDs of RIG-I. In another embodiment, the polypeptide is a MDA5 polypeptide, such as a full length MDA5 polypeptide or a portion of MDA5 such as one or more CARDs of MDA5. A “residue” refers to an amino acid or amino acid mimetic incorporated in a polypeptide by an amide bond or amide bond mimetic. A polypeptide has an amino terminal (N-terminal) end and a carboxy terminal (C-terminal) end. “Polypeptide” is used interchangeably with peptide or protein, and is used interchangeably herein to refer to a polymer of amino acid residues.

Preventing, Inhibiting or Treating a Disease: Inhibiting the full development of a disease or condition, for example, in a subject who is at risk for a disease such as viral infection, for example infection with an RNA virus, for example a dsRNA virus or a ssRNA virus such as an influenza virus. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease. A “prophylactic” treatment is a treatment administered to a subject who does not exhibit signs of a disease or exhibits only early signs for the purpose of decreasing the risk of developing pathology. A “prophylactic” includes vaccination against the disease or condition, for example, vaccination against a viral infection.

Purified: The term “purified” (for example, with respect to an adenovirus vector or recombinant adenovirus) does not require absolute purity; rather, it is intended as a relative term. Thus, for example, a purified nucleic acid is one in which the nucleic acid is more enriched than the nucleic acid in its natural environment within a cell. Similarly, a purified peptide preparation is one in which the peptide or protein is more enriched than the peptide or protein is in its natural environment within a cell. In one embodiment, a preparation is purified such that the specified component represents at least 50% (such as, but not limited to, 70%, 80%, 90%, 95%, 98% or 99%) of the total preparation by weight or volume.

Replication defective adenovirus vector: An adenovirus vector that does not have the genes to replicate.

RIG-I: An intracellular DExD/H box-RNA helicase having a C-terminal domain that binds double-stranded RNA (dsRNA) and two N-terminal caspase recruitment domain (CARD) domains. RIG-1 senses intracellular viral double stranded RNA and stimulates the expression of type-1 interferons, such as interferon-β (IFN-β), and thus promotes the response to viral infection. RIG-I recognizes the dsRNA of several negative-sense ssRNA viruses (including influenza virus) and a positive-sense ssRNA virus, Japanese encephalitis virus (which is a flavivirus) among others.

Transformed: A transformed cell is a cell into which has been introduced a nucleic acid molecule by molecular biology techniques. As used herein, the term transformation encompasses all techniques by which a nucleic acid molecule might be introduced into such a cell, including transfection with viral vectors, transformation with plasmid vectors, and introduction of DNA by electroporation, lipofection, and particle gun acceleration.

Vaccine: A vaccine is a pharmaceutical composition that elicits a prophylactic or therapeutic immune response in a subject. In some cases, the immune response is a protective immune response. Typically, a vaccine elicits an antigen-specific immune response to an antigen of a pathogen, for example to a virus. The vaccines described herein include adenovirus vectors or recombinant adenoviruses.

Vector: A nucleic acid molecule as introduced into a host cell, thereby producing a transformed host cell. Recombinant DNA vectors are vectors having recombinant DNA. A vector can include nucleic acid sequences that permit it to replicate in a host cell, such as an origin of replication. A vector can also include one or more selectable marker genes and other genetic elements known in the art. Viral vectors are recombinant DNA vectors having at least some nucleic acid sequences derived from one or more viruses. The term vector includes plasmids, linear nucleic acid molecules, and as described throughout adenovirus vectors and adenoviruses. The term adenovirus vector is utilized herein to refer to nucleic acids including one or more components of an adenovirus that replicate to generate viral particles in host cells (infectious). An adenovirus includes nucleic acids that encode at least a portion of the assembled virus. Thus, in many circumstances, the terms can be used interchangeably. Accordingly, as used herein the terms are used with specificity to facilitate understanding and without the intent to limit the embodiment in any way.

Virus: Microscopic infectious organism that reproduces inside living cells. A virus consists essentially of a core of nucleic acid surrounded by a protein coat, and has the ability to replicate only inside a living cell, for example as a viral infection. “Viral replication” is the production of additional virus by the occurrence of at least one viral life cycle. A virus, for example during a viral infection, may subvert the host cells' normal functions, causing the cell to behave in a manner determined by the virus. For example, a viral infection may result in a cell producing a cytokine, or responding to a cytokine, when the uninfected cell does not normally do so.

An RNA virus is a virus which belongs to either Group III, Group IV or Group V of the Baltimore classification system (see, Luria, et al. General Virology, 3rd Edn. John Wiley & Sons, New York, p2 of 578, 1978). RNA viruses possess ribonucleic acid (RNA) as their genetic material and typically do not replicate using a DNA intermediate. The nucleic acid is usually single-stranded RNA (ssRNA) but can occasionally be double-stranded RNA (dsRNA). Group III viruses include dsRNA viruses, for example viruses from: Birnaviridae, Chrysoviridae, Cystoviridae, Hypoviridae, Partitiviridae, Reoviridae (such as Rotavirus), and Totiviridae among others. Group IV includes the positive sense ssRNA viruses and includes for example viruses from: Nidovirales, Arteriviridae, Coronaviridae (such as Coronavirus and SARS), Roniviridae, Astroviridae, Barnaviridae, Bromoviridae, Caliciviridae, Closteroviridae, Comoviridae, Dicistroviridae, Flaviviridae (such as Yellow fever virus, West Nile virus, Hepatitis C virus, and Dengue fever virus), Flexiviridae, Hepeviridae (such as Hepatitis E virus), Leviviridae, Luteoviridae, Marnaviridae, Narnaviridae, Nodaviridae Picornaviridae (such as Poliovirus, the common cold virus, and Hepatitis A virus), Potyviridae, Sequiviridae, Tetraviridae, Togaviridae (such as Rubella virus and Ross River virus), Tombusviridae, and Tymoviridae among others. Group V viruses are negative sense ssRNA viruses and include for example viruses from: Bornaviridae (such as Borna disease virus), Filoviridae (such as Ebola virus and Marburg virus, Paramyxoviridae (such as Measles virus, and Mumps virus), Rhabdoviridae (such as Rabies virus), Arenaviridae (such as Lassa fever virus), Bunyaviridae (such as Hantavirus), and Orthomyxoviridae (such as Influenza viruses) among others.

The term “adenovirus” as used herein is intended to encompass all adenoviruses, including the Mastadenovirus and Aviadenovirus genera. To date, at least forty-seven human serotypes of adenoviruses have been identified (see, for example, Fields et al., VIROLOGY, volume 2, chapter 67 (3d ed., Lippincont-Raven Publishers). Adenoviruses are linear double-stranded DNA viruses approximately 36 kb in size. Their genome includes an inverted sequence (ITR) at each end, an encapsidation sequence, early genes and late genes. The main early genes are contained in the E1, E2, E3 and E4 regions. Among them, the genes contained in the E1 region (E1a and E1b, in particular) are believed necessary for viral replication. The E4 and L5 regions, for example, are involved in viral propagation, and the main late genes are contained in the L1 to L5 regions. For example, the human Ad5 adenovirus genome has been sequenced completely and the sequence is available (see, for example, GENBANK® Accession No. M73260). Similarly, portions, or in some cases the whole, of the genome of human and non-human adenoviruses of different serotypes (Ad2, Ad3, Ad7, Ad12, and the like) have also been sequenced.

“Influenza viruses” have a segmented single-stranded (negative or antisense) genome. The influenza virion consists of an internal ribonucleoprotein core containing the single-stranded RNA genome and an outer lipoprotein envelope lined by a matrix protein. The segmented genome of influenza A consists of eight linear RNA molecules that encode ten polypeptides. Two of the polypeptides, HA and NA, include the primary antigenic determinants or epitopes required for a protective immune response against influenza. Based on the antigenic characteristics of the HA and NA proteins, influenza strains are classified into subtypes. “Avian influenza” usually refers to influenza A viruses found chiefly in birds. Recent outbreaks of avian influenza in Asia have been categorized as H5N1, H7N7 and H9N2 based on their HA and NA phenotypes. These subtypes have proven highly infectious in poultry and have been able to jump the species barrier to directly infect humans causing significant morbidity and mortality.

Hemagglutinin (HA) is a surface glycoprotein which projects from the lipoprotein envelope and mediates attachment to and entry into cells. The HA protein is approximately 566 amino acids in length, and is encoded by an approximately 1780 base polynucleotide sequence of segment 4 of the genome. Polynucleotide and amino acid sequences of HA (and other influenza antigens) isolated from recent, as well as historic, avian influenza strains can be found, for example, in the GENBANK® database (available on the world wide web at ncbi.nlm.nih.gov/entrez). For example recent avian H5 subtype HA sequences include: AY075033, AY075030, AY818135, AF046097, AF046096, and AF046088; recent H7 subtype HA sequences include: AJ704813, AJ704812, and Z47199; and, recent avian H9 subtype HA sequences include: AY862606, AY743216, and AY664675. One of ordinary skill in the art will appreciate that essentially any previously described or newly discovered avian HA antigen can be utilized in the compositions and methods described herein. Typically, the appropriate HA sequence or sequences are selected based on circulating or predicted avian and/or pandemic HA subtypes, for example, as recommended by the World Health Organization. Pandemic influenza typically refers to a new influenza virus for which people have little or no natural immunity. Pandemic influenza can sweep across the country and around the world in very short time.

In addition to the HA antigen, which is the predominant target of neutralizing antibodies against influenza, the neuraminidase (NA) envelope glycoprotein is also a target of the protective immune response against influenza. NA is an approximately 450 amino acid protein encoded by an approximately 1410 nucleotide sequence of influenza genome segment 6. Recent pathogenic avian strains of influenza have belonged to the N1, N7 and N2 subtypes. Exemplary NA polynucleotide and amino acid sequences include, for example, N1: AY651442, AY651447, and AY651483; N7: AY340077, AY340078 and AY340079; and, N2: AY664713, AF508892 and AF508588. Additional NA antigens can be selected from among previously described or newly discovered NA antigens based on circulating and/or predicted avian and/or pandemic NA subtypes.

The remaining segments of the influenza genome encode the internal proteins. While immunization with internal proteins alone does not give rise to a substantially protective neutralizing antibody response, T-cell responses to one or more of the internal proteins can significantly contribute to protection against influenza infection. Compared to the polymorphic HA and NA antigens, the internal proteins are more highly conserved between strains, and between subtypes. Thus, a T cell receptor elicited by exposure to an internal protein of an avian or human subtype of influenza binds to the comparable internal protein of other avian and human subtypes.

PB2 is a 759 amino acid polypeptide which is one of the three proteins which comprise the RNA-dependent RNA polymerase complex. PB2 is encoded by approximately 2340 nucleotides of the influenza genome segment 1. The remaining two polymerase proteins, PB1, a 757 amino acid polypeptide, and PA, a 716 amino acid polypeptide, are encoded by a 2341 nucleotide sequence and a 2233 nucleotide sequence (segments 2 and 3), respectively.

Segment 5 consists of 1565 nucleotides encoding a 498 amino acid nucleoprotein (NP) protein that forms the nucleocapsid. Segment 7 consists of a 1027 nucleotide sequence encoding a 252 amino acid M1 protein, and a 96 amino acid M2 protein, which is translated from a spliced variant of the M RNA. Segment 8 consists of an 890 nucleotide sequence encoding two nonstructural proteins, NS1 and NS2.

Of these proteins, the M (M1 and M2) and NP proteins are most likely to elicit protective humoral and/or cellular T cell responses. Accordingly, while any of the internal proteins can be included (for example, in addition to one or more avian HA and/or NA antigens) in the compositions and methods described herein, adenovirus vectors and adenoviruses commonly also include one or more of M1, M2 and/or NP proteins. As responses against internal protein(s) of one strain of virus tend to interact with internal protein(s) of other strains of influenza, the internal protein can be selected from essentially any avian and/or human strain. For example, the internal protein(s) can be selected from avian H5N1, H7N7 and/or H9N2 strains. Alternatively, the internal protein(s) can be selected from human H3N2, H1N1, and/or H2N2. Exemplary internal protein polynucleotide and amino acid sequences can be found, for example, in GENBANK®. For example, H3N2 M and NP nucleic acids and proteins are represented by Accession Nos. AF255370 and CY000756, respectively. The internal proteins of influenza are more conserved between strains and tend to elicit a cross-reactive T cell response that contributes to the protective immune response against influenza. Methods of producing adenovirus vectors and adenoviruses containing influenza antigens can be found in International Patent Application No. PCT/US2006/013384, which is incorporated by reference herein in its entirety.

### III. Description of Several Embodiments

The cytosolic proteins retinoic acid inducible gene I (RIG-I) and melanoma differentiation-associated gene 5 (MDA5) initiate IFN-I production in response to a viral infection, such as an infection of a subject by ssRNA viruses, for example influenza virus, Japanese encephalitis virus, hepatitis C virus, paramyxoviruses, and picornavirus among others. It is believed that the C-terminal helicase domains of RIG-I or MDA5 recognize viral dsRNA either produced during viral infection, from RNA secondary structure present in the single stranded RNA of ssRNA viruses as well as ssRNA containing 5′ phosphates from ssRNA viruses. The recognition of viral RNA is believed to lead to a structural change in RIG-I or MDA5 that allows the N-terminal CARDs of RIG-I or MDA5 to initiate IFN-I production through the interaction with heterologous CARDs from other proteins. In the absence of dsRNA, a liberated N-terminal portion of RIG-I or MDA5 containing CARDs can constitutively stimulate the production of IFN, thereby activating and/or stimulating a subject's immune system. As disclosed herein, it was discovered that the N-terminal portion of RIG-I, containing the two RIG-I CARDs, inhibited viral replication in lung epithelial cells. This finding demonstrates for the first time that the CARDs from RIG-I and MDA5 can be used to treat viral infections.

Provided herein in various embodiments are adenoviral vectors that contain nucleic acid sequences encoding CARDs. The adenoviral vectors that contain nucleic acid sequences encoding CARDs are capable of stimulating INF-I production in the absence of dsRNA, thus, the disclosed adenoviral vectors do not contain a nucleic acid sequence that encodes a helicase domain, such as helicase domains from RIG-I or MDA5. The disclosed adenoviral vectors are useful in enhancing immunogenic responses in vertebrate animals (such as birds or mammals, for example primates, such as humans) to pathogens, such as viral pathogens. The disclosed adenoviral vectors are particularly useful in treating and/or inhibiting viral infections, such as infections from dsRNA viruses and/or ssRNA viruses such as Japanese encephalitis virus, and hepatitis C virus, paramyxoviruses, Newcastle disease virus, picornavirus and influenza virus (for example, influenza A, influenza B, pandemic strains and/or avian strains of influenza) among others.

### A. CARD Polypeptides and Nucleic Acids Encoding CARD

The present disclosure relates to polypeptides that contain CARDs and nucleic acid molecules encoding CARD containing polypeptides. The disclosed nucleic acid molecules are capable of expressing CARDs in a cell, such as a cell from a subject, for example a human subject. In several embodiments these polypeptides and nucleic acid molecules stimulate and/or enhance an immune response to a virus and/or a viral infection.

In some embodiments, the CARDs are derived from human RIG-I and/or human MDA5. The human forms of RIG-I and MDA5 both contain two N-terminal CARDs. An exemplary amino acid sequence of RIG-I is set forth below as SEQ ID NO:1 (GENBANK® ACCESSION NUMBER NP_{—}055129). The first CARD of RIG-I spans from about residue 1 to about residue 87 of the amino acid sequence set forth as SEQ ID NO:1. The second CARD of RIG-I spans from about residue 92 to about residue 172 of the amino acid sequence set forth as SEQ ID NO:1. The C-terminal helicase domain of RIG-I spans from about residue 610 to about residue 776 of the amino acid sequence set forth as SEQ ID NO:1. The ATP binding domain of RIG-I spans from about residue 251 to about residue 430 of the amino acid sequence set forth as SEQ ID NO:1.

(SEQ ID NO: 1)

mtteqrrslqafqdyirktldptyilsymapwfreeevqyiqaeknnkgp
meaatlflkfllelqeegwfrgfldaldhagysglyeaieswdfkkiekl
eeyrlllkrlqpefktriiptdiisdlseclinqeceeilqicstkgmma
gaeklvecllrsdkenwpktlklalekernkfselwivekgikdvetedl
edkmetsdiqifyqedpecqnlsenscppsevsdtnlyspfkprnyqlel
alpamkgkntiicaptgcgktfvsllicehhlkkfpqgqkgkvvffanqi
pvyeqqksvfskyferhgyrvtgisgataenvpveqivenndiiiltpqi
lvnnlkkgtipslsiftlmifdechntskqhpynmimfnyldqklggssg
plpqvigltasvgvgdakntdealdyicklcasldasviatvkhnleele
qvvykpqkffrkvesrisdkfkyiiaqlmrdteslakrickdlenlsqiq
nrefgtqkyeqwivtvqkacmvfqmpdkdeesrickalflytshlrkynd
aliiseharmkdaldylkdffsnvraagfdeieqdltqrfeeklqelesv
srdpsnenpkledlcfilqeeyhlnpetitilfvktralvdalknwiegn
pklsflkpgiltgrgktnqntgmtlpaqkcildafkasgdhniliatsva
degidiaqcnlvilyeyvgnvikmiqtrgrgrargskcflltsnagviek
eqinmykekmmndsilrlqtwdeavfrekilhiqthekfirdsqekpkpv
pdkenkkllcrkckalacytadvrvieechytvlgdafkecfvsrphpkp
kqfssfekrakifcarqncshdwgihvkyktfeipvikiesfvvediatg
vqtlyskwkdfhfekipfdpaemsk

An exemplary nucleic acid sequence encoding a RIG-I polypeptide is set forth below as SEQ ID NO:2. Multiple additional nucleic acid sequences that encode the RIG-I polypeptide are known in view of the degeneracy of the genetic code. The first CARD of RIG-I is encoded by the nucleic acid sequence from about nucleotide 1 to about nucleotide 261 of SEQ ID NO:2. The second CARD of RIG-I is encoded by the nucleic acid sequence from about nucleotide 274 to about nucleotide 516 of SEQ ID NO:2.

(SEQ ID NO: 2)

atgaccaccgagcagcgacgcagcctgcaagccttccaggattatatccg
gaagaccctggaccctacctacatcctgagctacatggccccctggttta
gggaggaagaggtgcagtatattcaggctgagaaaaacaacaagggccca
atggaggctgccacactttttctcaagttcctgttggagctccaggagga
aggctggttccgtggctttttggatgccctagaccatgcaggttattctg
gactttatgaagccattgaaagttgggatttcaaaaaaattgaaaagttg
gaggagtatagattacttttaaaacgtttacaaccagaatttaaaaccag
aattatcccaaccgatatcatttctgatctgtctgaatgtttaattaatc
aggaatgtgaagaaattctacagatttgctctactaaggggatgatggca
ggtgcagagaaattggtggaatgccttctcagatcagacaaggaaaactg
gcccaaaactttgaaacttgctttggagaaagaaaggaacaagttcagtg
aactgtggattgtagagaaaggtataaaagatgttgaaacagaagatctt
gaggataagatggaaacttctgacatacagattttctaccaagaagatcc
agaatgccagaatcttagtgagaattcatgtccaccttcagaagtgtctg
atacaaacttgtacagcccatttaaaccaagaaattaccaattagagctt
gctttgcctgctatgaaaggaaaaaacacaataatatgtgctcctacagg
ttgtggaaaaacctttgtttcactgcttatatgtgaacatcatcttaaaa
aattcccacaaggacaaaaggggaaagttgtcttttttgcgaatcagatc
ccagtgtatgaacagcagaaatctgtattctcaaaatactttgaaagaca
tgggtatagagttacaggcatttctggagcaacagctgagaatgtcccag
tggaacagattgttgagaacaatgacatcatcattttaactccacagatt
cttgtgaacaaccttaaaaagggaacgattccatcactatccatctttac
tttgatgatatttgatgaatgccacaacactagtaaacaacacccgtaca
atatgatcatgtttaattatctagatcagaaacttggaggatcttcaggc
ccactgccccaggtcattgggctgactgcctcggttggtgttggggatgc
caaaaacacagatgaagccttggattatatctgcaagctgtgtgcttctc
ttgatgcgtcagtgatagcaacagtcaaacacaatctggaggaactggag
caagttgtttataagccccagaagtttttcaggaaagtggaatcacggat
tagcgacaaatttaaatacatcatagctcagctgatgagggacacagaga
gtctggcaaagagaatctgcaaagacctcgaaaacttatctcaaattcaa
aatagggaatttggaacacagaaatatgaacaatggattgttacagttca
gaaagcatgcatggtgttccagatgccagacaaagatgaagagagcagga
tttgtaaagccctgtttttatacacttcacatttgcggaaatataatgat
gccctcattatcagtgagcatgcacgaatgaaagatgctctggattactt
gaaagacttcttcagcaatgtccgagcagcaggattcgatgagattgagc
aagatcttactcagagatttgaagaaaagctgcaggaactagaaagtgtt
tccagggatcccagcaatgagaatcctaaacttgaagacctctgcttcat
cttacaagaagagtaccacttaaacccagagacaataacaattctctttg
tgaaaaccagagcacttgtggacgctttaaaaaattggattgaaggaaat
cctaaactcagttttctaaaacctggcatattgactggacgtggcaaaac
aaatcagaacacaggaatgaccctcccggcacagaagtgtatattggatg
cattcaaagccagtggagatcacaatattctgattgccacctcagttgct
gatgaaggcattgacattgcacagtgcaatcttgtcatcctttatgagta
tgtgggcaatgtcatcaaaatgatccaaaccagaggcagaggaagagcaa
gaggtagcaagtgcttccttctgactagtaatgctggtgtaattgaaaaa
gaacaaataaacatgtacaaagaaaaaatgatgaatgactctattttacg
ccttcagacatgggacgaagcagtatttagggaaaagattctgcatatac
agactcatgaaaaattcatcagagatagtcaagaaaaaccaaaacctgta
cctgataaggaaaataaaaaactgctctgcagaaagtgcaaagccttggc
atgttacacagctgacgtaagagtgatagaggaatgccattacactgtgc
ttggagatgcttttaaggaatgctttgtgagtagaccacatcccaagcca
aagcagttttcaagttttgaaaaaagagcaaagatattctgtgcccgaca
gaactgcagccatgactggggaatccatgtgaagtacaagacatttgaga
ttccagttataaaaattgaaagttttgtggtggaggatattgcaactgga
gttcagacactgtactcgaagtggaaggactttcattttgagaagatacc
atttgatccagcagaaatgtccaaatga

An exemplary amino acid sequence of MDA5 is set forth below as SEQ ID NO:3 (GENBANK® Accession No. AAG34368). The first CARD of MDA5 spans from about residue 7 to about residue 97 of the amino acid sequence set forth as SEQ ID NO:3. The second CARD of MDA5 spans about residue 110 to about residue 190 of the amino acid sequence set forth as SEQ ID NO:3. The C-terminal helicase domain of MDA5 spans from about residue 700 to about residue 882 of the amino acid sequence set forth as SEQ ID NO:3. The ATP binding domain of MDA5 spans from about residue 316 to about residue 509 of the amino acid sequence set forth as SEQ ID NO:3.

(SEQ ID NO: 3)

msngystdenfryliscfrarvkmyiqvepvldyltflpaevkeqiqrtv
atsgnmqavelllstlekgvwhlgwtrefvealrrtgsplaarymnpelt
dlpspsfenahdeylqllnllqptlvdkllvrdvldkcmeeelltiedrn
riaaaenngnesgvrellkrivqkenwfsaflnvlrqtgnnelvqeltgs
dcsesnaeienlsqvdgpqveeqllsttvqpnlekevwgmennssessfa
dssvvsesdtslaegsvscldeslghnsnmgsdsgtmgsdsdeenvaara
spepelqlrpyqmevaqpalegknhiiclptgsgktrvavyiakdhldkk
kkasepgkvivlvnkvllveqlfrkefqpflkkwyrviglsgdtqlkisf
pevvkscdiiistaqilensllnlengedagvqlsdfsliiidechhtnk
eavynnimrhylmqklknnrlkkenkpviplpqilgltaspgvggatkqa
kaeehilklcanldaftiktvkenldqlknqiqepckkfaiadatredpf
keklleimtriqtycqmspmsdfgtqpyeqwaiqmekkaakkgnrkervc
aehlrkynealqindtirmidaythletfyneekdkkfavieddsdeggd
deycdgdededdlkkplkldetdrflmtlffennkmlkrlaenpeyenek
ltklrntimeqytrteesargiiftktrqsayalsqwitenekfaevgvk
ahhligaghssefkpmtqneqkeviskfrtgkinlliattvaeegldike
cniviryglvtneiamvqargraradestyvlvahsgsgviehetvndfr
ekmmykaihcvqnmkpeeyahkilelqmqsimekkmktkrniakhyknnp
slitflckncsvlacsgedihviekmhhvnmtpefkelyivrenkalqkk
cadyqingeiickcgqawgtmmvhkgldlpclkirnfvvvfknnstkkqy
kkwvelpitfpnldysecclfsded

An exemplary nucleic acid sequence encoding an MDA5 polypeptide is set forth below as SEQ ID NO:4. Multiple additional nucleic acid sequences that encode the MDA5 polypeptide are known in view of the degeneracy of the genetic code. The first CARD of MDA5 is encoded by the nucleic acid sequence from about nucleotide 19 to about nucleotide 291 of SEQ ID NO:4. The second CARD of MDA5 is encoded by the nucleic acid sequence from about 328 to about 570 of SEQ ID NO:4.

(SEQ ID NO: 4)

atgtcgaatgggtattccacagacgagaatttccgctatctcatctcgtg
cttcagggccagggtgaaaatgtacatccaggtggagcctgtgctggact
acctgacctttctgcctgcagaggtgaaggagcagattcagaggacagtc
gccacctccgggaacatgcaggcagttgaactgctgctgagcaccttgga
gaagggagtctggcaccttggttggactcgggaattcgtggaggccctcc
ggagaaccggcagccctctggccgcccgctacatgaaccctgagctcacg
gacttgccctctccatcgtttgagaacgctcatgatgaatatctccaact
gctgaacctccttcagcccactctggtggacaagcttctagttagagacg
tcttggataagtgcatggaggaggaactgttgacaattgaagacagaaac
cggattgctgctgcagaaaacaatggaaatgaatcaggtgtaagagagct
actaaaaaggattgtgcagaaagaaaactggttctctgcatttctgaatg
ttcttcgtcaaacaggaaacaatgaacttgtccaagagttaacaggctct
gattgctcagaaagcaatgcagagattgagaatttatcacaagttgatgg
tcctcaagtggaagagcaacttctttcaaccacagttcagccaaatctgg
agaaggaggtctggggcatggagaataactcatcagaatcatcttttgca
gattcttctgtagtttcagaatcagacacaagtttggcagaaggaagtgt
cagctgcttagatgaaagtcttggacataacagcaacatgggcagtgatt
caggcaccatgggaagtgattcagatgaagagaatgtggcagcaagagca
tccccggagccagaactccagctcaggccttaccaaatggaagttgccca
gccagccttggaagggaagaatatcatcatctgcctccctacagggagtg
gaaaaaccagagtggctgtttacattgccaaggatcacttagacaagaag
aaaaaagcatctgagcctggaaaagttatagttcttgtcaataaggtact
gctagttgaacagctcttccgcaaggagttccaaccatttttgaagaaat
ggtatcgtgttattggattaagtggtgatacccaactgaaaatatcattt
ccagaagttgtcaagtcctgtgatattattatcagtacagctcaaatcct
tgaaaactccctcttaaacttggaaaatggagaagatgctggtgttcaat
tgtcagacttttccctcattatcattgatgaatgtcatcacaccaacaaa
gaagcagtgtataataacatcatgaggcattatttgatgcagaagttgaa
aaacaatagactcaagaaagaaaacaaaccagtgattccccttcctcaga
tactgggactaacagcttcacctggtgttggaggggccacgaagcaagcc
aaagctgaagaacacattttaaaactatgtgccaatcttgatgcatttac
tattaaaactgttaaagaaaaccttgatcaactgaaaaaccaaatacagg
agccatgcaagaagtttgccattgcagatgcaaccagagaagatccattt
aaagagaaacttctagaaataatgacaaggattcaaacttattgtcaaat
gagtccaatgtcagattttggaactcaaccctatgaacaatgggccattc
aaatggaaaaaaaagctgcaaaaaaaggaaatcgcaaagaacgtgtttgt
gcagaacatttgaggaagtacaatgaggccctacaaattaatgacacaat
tcgaatgatagatgcgtatactcatcttgaaactttctataatgaagaga
aagataagaagtttgcagtcatagaagatgatagtgatgagggtggtgat
gatgagtattgtgatggtgatgaagatgaggatgatttaaagaaaccttt
gaaactggatgaaacagatagatttctcatgactttattttttgaaaaca
ataaaatgttgaaaaggctggctgaaaacccagaatatgaaaatgaaaag
ctgaccaaattaagaaataccataatggagcaatatactaggactgagga
atcagcacgaggaataatctttacaaaaacacgacagagtgcatatgcgc
tttcccagtggattactgaaaatgaaaaatttgctgaagtaggagtcaaa
gcccaccatctgattggagctggacacagcagtgagttcaaacccatgac
acagaatgaacaaaaagaagtcattagtaaatttcgcactggaaaaatca
atctgcttatcgctaccacagtggcagaagaaggtctggatattaaagaa
tgtaacattgttatccgttatggtctcgtcaccaatgaaatagccatggt
ccaggcccgtggtcgagccagagctgatgagagcacctacgtcctggttg
ctcacagtggttcaggagttatcgaacatgagacagttaatgatttccga
gagaagatgatgtataaagctatacattgtgttcaaaatatgaaaccaga
ggagtatgctcataagattttggaattacagatgcaaagtataatggaaa
agaaaatgaaaaccaagagaaatattgccaagcattacaagaataaccca
tcactaataactttcctttgcaaaaactgcagtgtgctagcctgttctgg
ggaagatatccatgtaattgagaaaatgcatcacgtcaatatgaccccag
aattcaaggaactttacattgtaagagaaaacaaagcactgcaaaagaag
tgtgccgactatcaaataaatggtgaaatcatctgcaaatgtggccaggc
ttggggaacaatgatggtgcacaaaggcttagatttgccttgtctcaaaa
taaggaattttgtagtggttttcaaaaataattcaacaaagaaacaatac
aaaaagtgggtagaattacctatcacatttcccaatcttgactattcaga
atgctgtttatttagtgatgaggattag

In some embodiments, the CARD containing polypeptides contain an amino acid sequence that is at least 95% identical to the amino acid sequence set forth as residues 1-87 of SEQ ID NO:1, such as at least 95%, at least 96%, at least 97%, at least 98%, at least 99%, or 100% identical to the amino acid sequence set forth as residues 1-87 of SEQ ID NO:1. In some embodiments, the CARD containing polypeptides contain an amino acid sequence that is at least 95% identical to the amino acid sequence set forth as residues 92-172 of SEQ ID NO:1, such as at least 95%, at least 96%, at least 97%, at least 98%, at least 99%, or 100% identical to the amino acid sequence set forth as residues 92-172 of SEQ ID NO:1. In some embodiments, the CARD containing polypeptides contain an amino acid sequence that is at least 95% identical to the amino acid sequence set forth as residues 1-284 of SEQ ID NO:1, such as at least 95%, at least 96%, at least 97%, at least 98%, at least 99%, or 100% identical to the amino acid sequence set forth as residues 1-284 of SEQ ID NO:1. In some embodiments, the CARD containing polypeptides contain an amino acid sequence that is at least 95% identical to the amino acid sequence set forth as residues 7-97 of SEQ ID NO:3, such as at least 95%, at least 96%, at least 97%, at least 98%, at least 99%, or 100% identical to the amino acid sequence set forth as residues 7-97 of SEQ ID NO:3. In some embodiments, the CARD containing polypeptides contain an amino acid sequence that is at least 95% identical to the amino acid sequence set forth as residues 110-190 of SEQ ID NO:3, such as at least 95%, at least 96%, at least 97%, at least 98%, at least 99%, or 100% identical to the amino acid sequence set forth as residues 110-190 of SEQ ID NO:3. In some embodiments, the CARD containing polypeptides contain an amino acid sequence that is at least 95% identical to the amino acid sequence set forth as residues 1-196 of SEQ ID NO:3, such as at least 95%, at least 96%, at least 97%, at least 98%, at least 99%, or 100% identical to the amino acid sequence set forth as residues 1-196 of SEQ ID NO:3.

In some instances it may be advantageous for the disclosed polypeptides to include multiple CARDs, such as 1, 2, 3, 4, or even more CARDs. For example, 1, 2 3, 4, or more CARDs from RIG-I and/or MDA5. In some embodiments, the disclosed polypeptides include multiple CARDs from RIG-I such as 1, 2, 3, 4, or more CARDs from RIG-I. In some embodiments, the disclosed polypeptides include multiple CARDs from MDA5 such as 1, 2, 3, 4, or more CARDs from MDA5. It may also be advantageous to include a CARD from MDA5 and a CARD from RIG-I. Thus in some embodiments, the disclosed polypeptides include at least one CARD from RIG-I (such as 1, 2, 3, 4, or more CARDs from RIG-I) and at least one CARD from MDA5 (such as 1, 2, 3, 4, or more CARDs from MDA5).

Also disclosed are nucleic acid molecules encoding these polypeptides. In some embodiments, the nucleic acid molecules include a nucleic acid sequence encoding an amino acid sequence at least 95% identical to the amino acids set forth as residues 1-87 of SEQ ID NO:1, such as at least 95%, at least 96%, at least 97%, at least 98%, at least 99%, or 100% identical to the amino acids set forth as residues 1-87 of SEQ ID NO:1. In some embodiments, the nucleic acid molecules include a nucleic acid sequence encoding an amino acid sequence at least 95% identical to the amino acids set forth as residues 92-172 of SEQ ID NO:1, such as at least 95%, at least 96%, at least 97%, at least 98%, at least 99%, or 100% identical to the amino acids set forth as 92-172 of SEQ ID NO:1. In some embodiments, the nucleic acid molecules include a nucleic acid sequence encoding an amino acid sequence at least 95% identical to the amino acids set forth as residues 1-284 of SEQ ID NO:1, such as at least 95%, at least 96%, at least 97%, at least 98%, at least 99%, or 100% identical to the amino acids set forth as residues 1-284 of SEQ ID NO:1. In some embodiments, the nucleic acid molecules include a nucleic acid sequence encoding an amino acid sequence at least 95% identical to the amino acids set forth as residues 7-97 of SEQ ID NO:3, such as at least 95%, at least 96%, at least 97%, at least 98%, at least 99%, or 100% identical to the amino acids set forth as residues 7-97 of SEQ ID NO:3. In some embodiments, the nucleic acid molecules include a nucleic acid sequence encoding an amino acid sequence at least 95% identical to the amino acids set forth as 110-190 of SEQ ID NO:3, such as at least 95%, at least 96%, at least 97%, at least 98%, at least 99%, or 100% identical to the amino acids set forth as 110-190 of SEQ ID NO:3. In some embodiments, the nucleic acid molecules include a nucleic acid sequence encoding an amino acid sequence at least 95% identical to the amino acids set forth as 1-196 of SEQ ID NO:3, such as at least 95%, at least 96%, at least 97%, at least 98%, at least 99%, or 100% identical to the amino acids set forth as 1-196 of SEQ ID NO:3.

In the context of the compositions and methods described herein, a nucleic acid sequence that encodes at least one CARD such as a CARD of RIG-I or MDA5, such as described above, is incorporated into a vector capable of expression in a host cell (for example an adenoviral vector), using established molecular biology procedures. For example nucleic acids, such as cDNAs, that encode at least one CARD can be manipulated with standard procedures such as restriction enzyme digestion, fill-in with DNA polymerase, deletion by exonuclease, extension by terminal deoxynucleotide transferase, ligation of synthetic or cloned DNA sequences, site-directed sequence-alteration via single-stranded bacteriophage intermediate or with the use of specific oligonucleotides in combination with PCR or other in vitro amplification.

Exemplary procedures sufficient to guide one of ordinary skill in the art through the production of vector capable of expression in a host cell (such as an adenoviral vector) that includes a polynucleotide sequence that encodes at least one CARD can be found for example in Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2003); and Ausubel et al., Short Protocols in Molecular Biology: A Compendium of Methods from Current Protocols in Molecular Biology, 4th ed., Wiley & Sons, 1999.

Typically, a polynucleotide sequence encoding at least one CARD is operably linked to transcriptional control sequences including, for example a promoter and a polyadenylation signal. A promoter is a polynucleotide sequence recognized by the transcriptional machinery of the host cell (or introduced synthetic machinery) that is involved in the initiation of transcription. A polyadenylation signal is a polynucleotide sequence that directs the addition of a series of nucleotides on the end of the mRNA transcript for proper processing and trafficking of the transcript out of the nucleus into the cytoplasm for translation.

Exemplary promoters include viral promoters, such as cytomegalovirus immediate early gene promoter (“CMV”), herpes simplex virus thymidine kinase (“tk”), SV40 early transcription unit, polyoma, retroviruses, papilloma virus, hepatitis B virus, and human and simian immunodeficiency viruses. Other promoters are isolated from mammalian genes, including the immunoglobulin heavy chain, immunoglobulin light chain, T-cell receptor, HLA DQ α and DQ β, β-interferon, interleukin-2, interleukin-2 receptor, MHC class II, HLA-DRα, β-actin, muscle creatine kinase, prealbumin (transthyretin), elastase I, metallothionein, collagenase, albumin, fetoprotein, β-globin, c-fos, c-HA-ras, insulin, neural cell adhesion molecule (NCAM), α1-antitrypsin, H2B (TH2B) histone, type I collagen, glucose-regulated proteins (GRP94 and GRP78), rat growth hormone, human serum amyloid A (SAA), troponin I (TNI), platelet-derived growth factor, and dystrophin, dendritic cell-specific promoters, such as CD11c, macrophage-specific promoters, such as CD68, Langerhans cell-specific promoters, such as Langerin, and promoters specific for keratinocytes, and epithelial cells of the skin and lung.

The promoter can be either inducible or constitutive. An inducible promoter is a promoter which is inactive or exhibits low activity except in the presence of an inducer substance. Examples of inducible promoters include, but are not limited to, MT II, MMTV, collagenase, stromelysin, SV40, murine MX gene, α-2-macroglobulin, MHC class I gene h-2 kb, HSP70, proliferin, tumor necrosis factor, or thyroid stimulating hormone gene promoter.

Typically, the promoter is a constitutive promoter that results in high levels of transcription upon introduction into a host cell in the absence of additional factors. Optionally, the transcription control sequences include one or more enhancer elements, which are binding recognition sites for one or more transcription factors that increase transcription above that observed for the minimal promoter alone.

It may be desirable to include a polyadenylation signal to effect proper termination and polyadenylation of the gene transcript. Exemplary polyadenylation signals have been isolated from bovine growth hormone, SV40 and the herpes simplex virus thymidine kinase genes. Any of these or other polyadenylation signals can be utilized in the context of the adenovirus vectors described herein.

It is understood that portions of the nucleic acid sequences encoding CARD containing polypeptides can be deleted as long as the polypeptides induce the production of IFN-I. For example, it may be desirable to delete one or more amino acids from the N-terminus, C-terminus, or both. Exemplary methods of determining the ability of the disclosed polypeptides to induce IFN-I are given in the examples below. It is also contemplated that the substitution of residues in the disclosed CARDs can be made, such that the ability of the CARD containing polypeptides retain the ability to induce IFN-I production. One of ordinary skill in the art can make the determination of which residues in the disclosed CARD containing polypeptides are tolerant of amino acid substitution for example be determining the sequence similarity between the individual CARDs of RIG-I or MDA5, and/or the sequence similarity between the CARDs of RIG-1 and MDA5. One of ordinary skill in the art would understand that regions of high sequence conservation are likely to be less tolerant of amino acid substitutions, while regions of relatively low sequence similarity would be perceived to be more tolerant of amino acid substitutions.

### B. Adenovirus Vectors Encoding CARD.

The present disclosure also relates to adenoviral vectors and adenoviruses containing nucleic acid molecules capable of expressing CARDs, such as CARDs from RIG-I and MDA5. The disclosed adenoviral vectors are capable of expressing CARDs in a cell, such as a cell of or from a subject, for example a human subject. Upon infection of a subject (or host) with recombinant adenoviruses, or introduction of a recombinant adenovirus vector, exogenous nucleic acids contained within the adenovirus genome are transcribed, and translated, by the host cell RNA polymerase and translational machinery. A polynucleotide sequence that encodes one or more CARDs, such as from RIG-I and/or MDA5, can be incorporated into an adenovirus vector and introduced into the cells of a subject where the polynucleotide sequence is transcribed and translated to produce the one or more CARDs. Thus, the adenoviruses disclosed herein are useful in stimulating and/or enhancing an immune response, such as an immune response to a virus, for example an RNA virus such as a dsRNA virus or a ssRNA virus (for example, an influenza virus such as influenza A, influenza B, pandemic strains and/or avian strains of influenza)

In some embodiments, the adenoviral vectors contain a nucleic acid sequence that encodes a CARD polypeptide that is at least 95% identical to the amino acid sequence set forth as residues 1-87 of SEQ ID NO:1, such as at least 95%, at least 96%, at least 97%, at least 98%, at least 99%, or 100% identical to the amino acid sequence set forth as residues 1-87 of SEQ ID NO:1. In some embodiments, the adenoviral vectors contain a nucleic acid sequence that encodes a CARD polypeptide that is at least 95% identical to the amino acid sequence set forth as residues 92-172 of SEQ ID NO:1, such as at least 95%, at least 96%, at least 97%, at least 98%, at least 99%, or 100% identical to the amino acid sequence set forth as residues 92-172 of SEQ ID NO:1. In some embodiments, the adenoviral vectors contain a nucleic acid sequence that encodes a polypeptide that is at least 95% identical to the amino acid sequence set forth as residues 1-284 of SEQ ID NO:1, such as at least 95%, at least 96%, at least 97%, at least 98%, at least 99%, or 100% identical to the amino acid sequence set forth as residues 1-284 of SEQ ID NO:1. In some embodiments, the adenoviral vectors contain a nucleic acid sequence that encodes a CARD polypeptide that is at least 95% identical to the amino acid sequence set forth as residues 7-97 of SEQ ID NO:3, such as at least 95%, at least 96%, at least 97%, at least 98%, at least 99%, or 100% identical to the amino acid sequence set forth as residues 7-97 of SEQ ID NO:3. In some embodiments, the adenoviral vectors contain a nucleic acid sequence that encodes a CARD polypeptide that is at least 95% identical to the amino acid sequence set forth as residues 110-190 of SEQ ID NO:3, such as at least 95%, at least 96%, at least 97%, at least 98%, at least 99%, or 100% identical to the amino acid sequence set forth as residues 110-190 of SEQ ID NO:3. In some embodiments, the adenoviral vectors contain a nucleic acid sequence that encodes a polypeptide that is at least 95% identical to the amino acid sequence set forth as residues 1-196 of SEQ ID NO:3, such as at least 95%, at least 96%, at least 97%, at least 98%, at least 99%, or 100% identical to the amino acid sequence set forth as residues 1-196 of SEQ ID NO:3.

In some instances it may be advantageous for the disclosed adenoviral vectors to include nucleic acid sequences that encode multiple CARDs, such as 1, 2, 3, 4, or even more CARDs. For example, the adenoviral vectors can include a nucleic acid sequence encoding 1, 2 3, 4, or more CARDs from RIG-I and/or MDA5. In some embodiments, the disclosed adenoviral vectors can contain at least one nucleic acid sequence encoding a CARD from RIG-I such as 1, 2, 3, 4, or more CARDs from RIG-I. In some embodiments, the disclosed adenoviral vectors can contain at least one nucleic acid sequence encoding a CARD from MDA5 such as 1, 2, 3, 4, or more CARDs from MDA5. It may also be advantageous to include a nucleic acid sequence encoding a CARD from MDA5 and a nucleic acid sequence encoding a CARD from RIG-I. Thus in some embodiments, the disclosed adenoviral vectors can contain at least one nucleic acid sequence encoding a CARD from RIG-I (such as 1, 2, 3, 4, or more CARDs from RIG-I) and at least one nucleic acid sequence encoding a CARD from MDA5 (such as 1, 2, 3, 4, or more CARDs from MDA5).

Nucleic acid vectors encoding adenoviruses are well-known in the art, and have been utilized for gene therapy and vaccine applications. Exemplary adenovirus vectors are described in Berkner, BioTechniques 6:616-629, 1988; Graham, Trend Biotechnol, 8:85-87, 1990; Graham & Prevec, in Vaccines: new approaches to immunological problems, Ellis (ed.), pp. 363-390, Butterworth-Heinemann, Woburn, 1992; Mittal et al., in Recombinant and Synthetic Vaccines, Talwar et al. (eds) pp. 362-366, Springer Verlag, New York, 1994; Rasmussen et al., Hum. Gene Ther. 16:2587-2599, 1999; Hitt & Graham, Adv. Virus Res. 55:479-505, 2000, Published U.S. Patent Application No. 2002/0192185, which are incorporated herein in their entirety to the extent that they are not inconsistent with the present disclosure.

In many instances the vectors are modified to make them replication defective, that is, incapable of replicating autonomously in the host cell, although in addition to such helper dependent adenovirus vectors, conditional replication competent and replication competent adenovirus vectors and viruses can also be used. Typically, the genome of a replication defective virus lacks at least some of the sequences necessary for replication of the virus in an infected cell. These regions may be either removed (wholly or partially), or rendered non-functional, or replaced by other sequences, and in particular by a sequence coding for a molecule of therapeutic interest, for example a CARD. Typically, the defective virus retains the sequences which are involved in encapsidation of viral particles.

Replication defective adenoviruses typically include a mutation, such as a deletion, in one or more of the E1 (E1a and/or E1b), E3 region, E2 region and/or E4 region have been deleted. The entire adenovirus genome except the ITR and packaging elements can be deleted and the resultant adenovirus vectors are known as helper-dependent vectors or “gutless” vectors. In some cases, heterologous DNA sequences are inserted in place of the deleted adenovirus sequence (Levrero et al., Gene 101:195-202, 1991; Ghosh-Choudhury et al., Gene 50:161-171, 1986). Other constructions contain a deletion in the E1 region and of a non-essential portion of the E4 region (WO 94/12649). Exemplary adenovirus vectors are also described in U.S. Pat. Nos. 6,328,958; 6,669,942; and 6,420,170, which are incorporated herein by reference.

Replication defective recombinant adenoviruses may be prepared in different ways, for example, in a competent cell line capable of complementing the entire defective functions essential for replication of the recombinant adenovirus. For example, adenovirus vectors can be produced in a complementation cell line (such as 293 cells) in which a portion of the adenovirus genome has been integrated. Such cells lines contain the left-hand end (approximately 11-12%) of the adenovirus serotype 5 (Ad5) genome, comprising the left-hand ITR, the encapsidation region and the E1 region, including E1a, E1b and a portion of the region coding for the pIX protein. This cell line is capable of complementing recombinant adenoviruses which are defective for the E1 region. Typically, expression of both E1A and E1B proteins is needed for E1 complementation.

Human adenovirus vectors are commonly utilized to introduce exogenous nucleic acids into human and animal cells and organisms. Adenoviruses exhibit broad host cell range, and can be utilized to infect human as well as non-human animals, including birds. Most commonly, the human adenovirus vectors are HAd5 vectors derived from adenovirus serotype 5 viruses. Due to the large size of the intact adenovirus genome, insertion of heterologous polynucleotide sequences is most conveniently performed using a shuttle plasmid. Sequences, such as those encoding CARDs are cloned into a shuttle vector which then undergoes homologous recombination with all or part of an adenovirus genome in cultured cells. Alternatively, homologous recombination can be done in bacteria to generate full length adenovirus vectors.

To avoid pre-existing host immunity to human adenoviruses, it may be desirable to use non-human adenovirus vectors. Human adenovirus is common in human populations. Thus, individuals may have circulating antibodies capable of neutralizing recombinant human adenovirus. To avoid undesirable neutralization, non-human adenovirus vectors can be used to circumvent any pre-existing immunity against human adenovirus.

Adenoviruses of animal origin are also capable of infecting human and non-human cells. Generally, adenoviruses of animal origin are incapable of propagating in human cells (see, international patent application WO 94/26914). Therefore, it may be desirable to use adenoviruses of animal origin in the context of the vectors and viruses described herein. The use of animal adenovirus vectors for human and animal vaccine development is discussed in detail in Bangari & Mittal, Vaccine 24:849-862, 2006, which is incorporated herein by reference. For example, animal adenovirus vectors can be selected from canine, bovine, murine (for example: MAV1, Beard et al., Virology 75:81, 1990), ovine, porcine, avian (for example chicken) or alternatively simian (for example SAV) adenoviruses. For example, bovine and porcine adenoviruses can be used to produce adenovirus vectors that express CARDs, including various bovine serotypes available from the ATCC (types 1 to 8) under the references ATCC VR-313, 314, 639-642, 768 and 769, and porcine adenovirus 5359. Exemplary bovine and porcine adenovirus vectors are described in published U.S. Patent Application No. 2002/0192185, and in U.S. Pat. Nos. 6,492,343 and 6,451,319, and the disclosures of these vectors are incorporated herein by reference. Additionally, simian adenoviruses of various serotypes, including SAd25, SAd22, SAd23 and SAd24, such as those referenced in the ATCC under the numbers VR-591-594, 941-943, 195-203, and the like, several serotypes (1 to 10) of avian adenovirus which are available in the ATCC, such as, the strains Phelps (ATCC VR-432), Fontes (ATCC VR-280), P7-A (ATCC VR-827), IBH-2A (ATCC VR-828), J2-A (ATCC VR-829), T8-A (ATCC VR-830), K-11 (ATCC VR-921) and strains referenced as ATCC VR-831 to 835, as well as murine adenoviruses FL (ATCC VR-550) and E20308 (ATCC VR-528), and ovine adenovirus type 5 (ATCC VR-1343) or type 6 (ATCC VR-1340) can be used.

Recombinant adenovirus expressing CARDs such as CARDs from RIG-I and/or MDA5 produced from the vectors described above are produced following introduction of the adenovirus vector into a suitable host cell. For example, in the case of replication defective vectors, the adenovirus vector is typically introduced into a cell line that complements the defective function. For example, E1 deficient virus can be grown in a cell line that complements E1 function due to expression of an introduced nucleic acid that encodes adenovirus E1 protein. Exemplary cell lines include both human and non-human cell lines that have been engineered to express an adenovirus E1 (such as E1A) proteins. For example, 293 cells that express adenovirus E1 proteins are commonly utilized to grow recombinant replication-defective adenoviruses that have a deletion of the E1 region. Additional suitable cell lines include MDBK-221, FBK-34, and fetal retinal cells of various origins. Specific examples of cell lines suitable for growing porcine and bovine recombinant adenovirus include FPRT-HE1-5 cells (Bangari & Mittal, Virus Res. 105:127-136, 2004) and FBRT-HE1 cells (van Olphen et al., Virology, 295:108-118, 2002), respectively. In certain embodiments, the cells express adenovirus E1 genes of more than one strain of virus, such as 2 or more different strains of virus with different species tropism. For example, the cells can express E1 genes of a human and a non-human (for example, pig and/or cow E1 genes). Those of ordinary skill in the art will readily be able to select or produce suitable additional or alternative cell lines that complement the replication functions of replication-defective adenovirus vectors. For example, any of the various mammalian cell lines disclosed herein (or known in the art) can be transfected with E1 and/or E3 genes of any of the strains of adenovirus, such as the exemplary strains disclosed herein, based on the particular adenovirus vector to be grown. For example, it is common to select E1 (and/or E3) genes that correspond to (that is, are from the same or a functionally similar strain) the same strain as the adenovirus vector. One of skill in the art will also appreciate that functionally similar variants (such as variants that share substantial sequence identity, or that specifically hybridize, for example, under high stringency conditions) to any of the exemplary adenovirus genes, can also be used to produce cell lines that support the growth of adenovirus vectors.

One common method for producing replication defective adenovirus vectors that incorporate exogenous nucleic acids is described in Ng et al., Hum. Gene Ther. 10:2667-2672, 1999, and Hum. Gene Ther. 11:693-699, 2000, which are incorporated herein in their entirety. Briefly, to produce a human adenovirus vector containing one or more CARDs (such as the CARDs from MDA5 and RIG-I), a polynucleotide sequence encoding one or more CARDs (for example, one or more CARDs from MDA5 and RIG-I) operably linked to a strong promoter (such as the CMV immediate early promoter) is inserted into a shuttle vector, such as pDC311. The pDC311 shuttle vector is a plasmid that contains the left end of HAd5 (approximately 4 kb) with a 3.1 kb E1 deletion, a loxP site for site specific recombination in the presence of Cre recombinase and an intact packaging signal (ψ). The shuttle vector is co-transfected into appropriate cells that express the Cre recombinase (such as 293 Cre cells) along with a plasmid that includes a replication defective HAd5 genome (for example, containing deletions in the E1 and/or E3 region genes) that lacks a packaging signal, and contains a loxP site. Homologous Cre mediated recombination results in the production of an adenovirus vector plasmid that encodes a replication defective adenovirus that expresses the inserted one or more CARDs.

Cells that express complementing replication function (such as E1 when the replication defective adenovirus vector lacks E1 function) can be transfected with a recombinant adenovirus vector according to standard procedures, such as electroporation, calcium phosphate precipitation, lipofection, etc., or infected with adenovirus at low infectivity (such as between 1-1000 p.f.u./cell). In some cases confluent monolayers of cells are utilized. The cells are then incubated (grown) for a period of time sufficient for expression and replication of adenovirus, and the cells are divided to maintain active growth and maximize virus recovery, prior to harvesting of recombinant adenovirus. Typically following several passages (for example, 2-5 passages), recombinant adenovirus is collected by lysing the cells to release the virus, and then concentrating the virus. Recombinant adenovirus can be concentrated by passing the lysate containing the virus over a density gradient (such as a CsCl density gradient). Following concentration the recombinant adenoviruses are typically dialyzed against a buffer (such as 10 mM Tris pH 8.0, 2 mM MgCl₂, 5% sucrose), titered and stored until use at −80° C. Methods for producing adenovirus at a large scale are described, for example, in published U.S. Patent Application No. 2003/0008375, which is incorporated herein by reference.

To elicit an immune response for a specified virus it may be advantageous to include a nucleic acid sequence that encodes a viral antigen in the disclosed adenoviral vectors, for example a nucleic acid sequence that encodes an internal protein or an external protein of a virus. Thus, the disclosed compositions are useful for generating protective immunity against a virus harboring the antigen included in the adenoviral vector. In some embodiments, the disclosed adenovirus vectors additionally contain a nucleic acid sequence that encodes at least one viral antigen. In some embodiments, the viral antigen is an internal protein or an external protein. For example an antigen can be a polypeptide expressed on the surface of a virus, such as a viral envelope protein. In some embodiments, the antigen is from an RNA virus, such as a dsRNA virus or a ssRNA virus. Examples of antigens include antigens selected from animal and human viral pathogens, such as influenza, RSV, HIV, Rotavirus, New Castle Disease Virus, Marek Disease Virus, Metapneumovirus, Parainfluenza viruses, Coronaviruses (including for example, SARS-CoV, HCoV-HKU1, HCoV-NL63 and TGEV), Hepatitis C virus, Flaviviruses (such as Dengue virus, Japanese Encephlitis virus, Kunjin virus, Yellow fever virus and West Nile virus), Filoviruses (such as Ebola virus and Marburg Virus), Caliciviruses (including Norovirus and Sapovirus), Human Papilloma Virus, Epstein Barr Virus, Cytomegalovirus, Varicella Zoster virus, and Herpes Simplex Virus among others. Non-limiting examples of antigens include: influenza antigen (such as hemagglutinin (HA), neuraminidase (NA) antigen, or an influenza internal protein, such as a PB1, PB2, PA, M1, M2, NP, NS1 or NS2 protein); RSV (Type A & B) F and G proteins; HIV p24, pol, gp41 and gp120; Rotavirus VP8 epitopes; New Castle Disease Virus F and HN proteins; Marek Disease Virus Glycoproteins: gB, gC, gD, gE, gH, gI, and gL; Metapneumovirus F and G proteins; Parainfluenza viruses F and HN proteins; Coronavirus (e.g. SARS-CoV, HCoV-HKU1, HCoV-NL63, TGEV) S, M and N proteins; Hepatitis C virus E1, E2 and core proteins; Dengue virus E1, E2 and core proteins; Japanese encephalitis virus E1, E2 and core proteins; Kunjin virus E1, E2 and core proteins; West Nile virus E1, E2 and core proteins; Yellow Fever virus E1, E2 and core proteins; Ebola virus and Marburg Virus structural glycoprotein; Norovirus and Sapovirus major capsid proteins; Human Papilloma Virus L1 protein; Epstein Barr Virus gp220/350 and EBNA-3A peptide; Cytomegalovirus gB glycoprotein, gH glycoprotein, pp65, IE1 (exon 4) and pp150; Varicella Zoster virus IE62 peptide and glycoprotein E epitopes; Herpes Simplex Virus Glycoprotein D epitopes, among many others.

In specific examples, the at least one viral antigen can be an influenza antigen, such as an HA antigen, an NA antigen, or a combination thereof. In some examples the influenza antigen is H5N1 strain antigen, an H7N7 strain antigen, or an H9N2 strain antigen. In some examples, the at least one viral antigen is an influenza internal protein, such as an M1 protein, an M2 protein, an NP protein, a PB1 protein, a PB2 protein, an NS1 protein, an NS2 protein, or a combination thereof. In some examples, the internal influenza protein is derived from influenza strain H1N1, H2N2, or H3N2. In some examples, viral antigen can be an influenza antigen such as an HA antigen or an NA antigen. In some examples, the influenza antigen is from influenza strain H5N, H7N7, or H9N2. In some embodiments, the disclosed adenovirus vectors additionally contain a nucleic acid sequence that encodes at least one influenza internal protein, such as an M1 protein, an M2 protein, an NP protein, a PB 1 protein, a PB2 protein, an NS1 protein, an NS2 protein, or a combination thereof. In some examples the internal protein is of an H1N1, H2N2 or H3N2 influenza strain. Exemplary antigens from influenza viral sources can be found for example in International Patent Application No. PCT/US2006/013384, which is incorporated by reference herein in its entirety. Flt3 ligand has been shown to expand the population of dendritic cells. Thus it can also be advantageous to include a nucleic acid sequence that encodes Flt3 ligand in the disclosed adenoviral vector.

### C. Therapeutic Compositions

The CARD polypeptides, nucleic acids encoding CARDs, recombinant adenovirus vectors and recombinant adenoviruses that express CARDs (such as CARDs from RIG-I and/or MDA5) disclosed herein can be administered in vitro, ex vivo or in vivo to a cell or subject. Generally, it is desirable to prepare the vectors or viruses as pharmaceutical compositions appropriate for the intended application. Accordingly, methods for making a medicament or pharmaceutical composition containing the polypeptides, nucleic acids, adenovirus vectors or adenoviruses described above are included herein. Typically, preparation of a pharmaceutical composition (medicament) entails preparing a pharmaceutical composition that is essentially free of pyrogens, as well as any other impurities that could be harmful to humans or animals. Typically, the pharmaceutical composition contains appropriate salts and buffers to render the components of the composition stable and allow for uptake of nucleic acids or virus by target cells.

Therapeutic compositions can be provided as parenteral compositions, such as for injection or infusion. Such compositions are formulated generally by mixing a disclosed therapeutic agent at the desired degree of purity, in a unit dosage injectable form (solution, suspension, or emulsion), with a pharmaceutically acceptable carrier, for example one that is non-toxic to recipients at the dosages and concentrations employed and is compatible with other ingredients of the formulation. In addition, a disclosed therapeutic agent can be suspended in an aqueous carrier, for example, in an isotonic buffer solution at a pH of about 3.0 to about 8.0, preferably at a pH of about 3.5 to about 7.4, 3.5 to 6.0, or 3.5 to about 5.0. Useful buffers include sodium citrate-citric acid and sodium phosphate-phosphoric acid, and sodium acetate/acetic acid buffers. The active ingredient, optionally together with excipients, can also be in the form of a lyophilisate and can be made into a solution prior to parenteral administration by the addition of suitable solvents. Solutions such as those that are used, for example, for parenteral administration can also be used as infusion solutions.

Pharmaceutical compositions can include an effective amount of the adenovirus vector or virus dispersed (for example, dissolved or suspended) in a pharmaceutically acceptable carrier or excipient. Pharmaceutically acceptable carriers and/or pharmaceutically acceptable excipients are known in the art and are described, for example, in Remington's Pharmaceutical Sciences, by E. W. Martin, Mack Publishing Co., Easton, Pa., 15th Edition (1975).

The nature of the carrier will depend on the particular mode of administration being employed. For example, parenteral formulations usually contain injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch or magnesium stearate. In addition, pharmaceutical compositions to be administered can contain minor amounts of non-toxic auxiliary substances, such as wetting or emulsifying agents, preservatives, and pH buffering agents and the like, for example sodium acetate or sorbitan monolaurate.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the pharmaceutical compositions is contemplated. Supplementary active ingredients also can be incorporated into the compositions. For example, certain pharmaceutical compositions can include the vectors or viruses in water, mixed with a suitable surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The pharmaceutical compositions (medicaments) can be prepared for use in prophylactic regimens (such as vaccines) and administered to human or non-human subjects (including birds, such as domestic fowl, for example, chickens, ducks, guinea fowl, turkeys and geese) to elicit an immune response against an influenza antigen (or a plurality of influenza antigens). Thus, the pharmaceutical compositions typically contain a pharmaceutically effective amount of the adenovirus vector or adenovirus.

In some cases the compositions are administered following infection to enhance the immune response, in such applications, the pharmaceutical composition is administered in a therapeutically effective amount. A therapeutically effective amount is a quantity of a composition used to achieve a desired effect in a subject. For instance, this can be the amount of the composition necessary to inhibit viral replication or to prevent or measurably alter outward symptoms of viral infection. When administered to a subject, a dosage will generally be used that will achieve target tissue concentrations (for example, in lymphocytes) that has been shown to achieve an in vitro or in vivo effect.

Administration of therapeutic compositions can be by any common route as long as the target tissue (typically, the respiratory tract) is available via that route. This includes oral, nasal, ocular, buccal, or other mucosal (such as rectal or vaginal) or topical administration. Alternatively, administration will be by orthotopic, intradermal subcutaneous, intramuscular, intraperitoneal, or intravenous injection routes. Such pharmaceutical compositions are usually administered as pharmaceutically acceptable compositions that include physiologically acceptable carriers, buffers or other excipients.

The pharmaceutical compositions can also be administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain about 100 mg of human serum albumin per milliliter of phosphate buffered saline. Other pharmaceutically acceptable carriers include aqueous solutions, non-toxic excipients, including salts, preservatives, buffers and the like may be used. Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components of the pharmaceutical composition are adjusted according to well known parameters.

Additional formulations are suitable for oral administration. Oral formulations can include excipients such as, pharmaceutical grades of mannitol, lactose, starch, magnesium stearate, sodium saccharine, cellulose, magnesium carbonate and the like. The compositions (medicaments) typically take the form of solutions, suspensions, aerosols or powders. Exemplary formulations can be found in U.S. Patent publication No. 20020031527, the disclosure of which is incorporated herein by reference. When the route is topical, the form may be a cream, ointment, salve or spray. Exemplary methods for intramuscular, intranasal and topical administration of the adenovirus vectors and adenoviruses described herein can be found, for example, in U.S. Pat. No. 6,716,823, which is incorporated herein by reference.

Optionally, the pharmaceutical compositions or medicaments can include a suitable adjuvant to increase the immune response. As used herein, an “adjuvant” is any potentiator or enhancer of an immune response. The term “suitable” is meant to include any substance which can be used in combination with the polypeptide, nucleic acid, adenovirus vector or adenovirus to augment the immune response, without producing adverse reactions in the vaccinated subject. Effective amounts of a specific adjuvant may be readily determined so as to optimize the potentiation effect of the adjuvant on the immune response of a vaccinated subject. For example, 0.5%-5% aluminum hydroxide (or aluminum phosphate) and MF-59 oil emulsion (0.5% polysorbate 80 and 0.5% sorbitan trioleate. Squalene (5.0%) aqueous emulsion) are adjuvants which have been favorably utilized in the context of influenza vaccines. Other adjuvants include mineral, vegetable or fish oil with water emulsions, incomplete Freund's adjuvant, E. coli J5, dextran sulfate, iron oxide, sodium alginate, Bacto-Adjuvant, certain synthetic polymers such as Carbopol (BF Goodrich Company, Cleveland, Ohio), poly-amino acids and co-polymers of amino acids, saponin, carrageenan, REGRESSIN™ (Vetrepharm, Athens, Ga.), AVRIDINE (N,N-dioctadecyl-N′,N′-bis(2-hydroxyethyl)-propanediamine), long chain polydispersed β (1,4) linked mannan polymers interspersed with O-acetylated groups (for example ACEMANNAN), deproteinized highly purified cell wall extracts derived from a non-pathogenic strain of Mycobacterium species (for example EQUIMUNE®, Vetrepharm Research Inc., Athens Ga.), Mannite monooleate, paraffin oil, or muramyl dipeptide. A suitable adjuvant can be selected by one of ordinary skill in the art.

An effective amount of the pharmaceutical composition is determined based on the intended goal, for example vaccination of a human or non-human subject. The appropriate dose will vary depending on the characteristics of the subject, for example, whether the subject is a human or non-human, the age, weight, and other health considerations pertaining to the condition or status of the subject, the mode, route of administration, and number of doses, and whether the pharmaceutical composition includes nucleic acids or viruses. Generally, the pharmaceutical compositions described herein are administered for the purpose of stimulating and/or enhancing an immune response for example, an immune response against a viral antigen.

A typical dose of a recombinant adenovirus is from 10 p.f.u. to 10¹⁵ p.f.u./administration. For example, a pharmaceutical composition can include from about 100 p.f.u. of a recombinant adenovirus, such as about 1000 p.f.u., about 10,000 p.f.u., or about 100,000 p.f.u. of each recombinant adenovirus in a single dosage. Optionally, a pharmaceutical composition can include at least about a million p.f.u. or more per administration. For example, in some cases it is desirable to administer about 10⁷, 10⁸, 10⁹ or 10¹⁰ p.f.u. of recombinant adenovirus that expresses a particular influenza antigen.

When administering an nucleic acid, such as an adenovirus vector, facilitators of nucleic acid uptake and/or expression can also be included, such as bupivacaine, cardiotoxin and sucrose, and transfection facilitating vehicles such as liposomal or lipid preparations that are routinely used to deliver nucleic acid molecules. Anionic and neutral liposomes are widely available and well known for delivering nucleic acid molecules (see, for example, Liposomes: A Practical Approach, RPC New Ed., IRL Press, 1990). Cationic lipid preparations are also well known vehicles for use in delivery of nucleic acid molecules. Suitable lipid preparations include DOTMA (N-[1-(2,3-dioleyloxy)propyl]-N,N,N-trimethylammonium chloride), available under the tradename LIPOFECTIN®, and DOTAP (1,2-bis(oleyloxy)-3-(trimethylammonio)propane). See, for example, Felgner et al., Proc. Natl. Acad. Sci. U.S.A. 84:7413-7416, 1987; Malone et al., Proc. Natl. Acad. Sci. U.S.A. 86:6077-6081, 1989; U.S. Pat. Nos. 5,283,185 and 5,527,928, and International Publication Nos. WO 90/11092, WO 91/15501 and WO 95/26356. These cationic lipids may preferably be used in association with a neutral lipid, for example DOPE (dioleyl phosphatidylethanolamine). Still further transfection-facilitating compositions that can be added to the above lipid or liposome preparations include spermine derivatives (see, for example, International Publication No. WO 93/18759) and membrane-permeabilizing compounds such as GALA, Gramicidine S and cationic bile salts (see, for example, International Publication No. WO 93/19768).

Alternatively, nucleic acids (such as adenovirus vectors) can be encapsulated, adsorbed to, or associated with, particulate carriers. Suitable particulate carriers include those derived from polymethyl methacrylate polymers, as well as PLG microparticles derived from poly(lactides) and poly(lactide-co-glycolides). See, for example, Jeffery et al., Pharm. Res. 10:362-368, 1993. Other particulate systems and polymers can also be used, for example, polymers such as polylysine, polyarginine, polyornithine, spermine, spermidine, as well as conjugates of these molecules.

The formulated vaccine compositions will typically include an adenoviral vector and/or an adenovirus. An appropriate effective amount can be readily determined by one of skill in the art. Such an amount will fall in a relatively broad range that can be determined through routine trials, for example within a range of about 10 μg to about 1 mg. However, doses above and below this range may also be found effective.

Nucleic acids such as adenoviral vectors can be coated onto carrier particles (for example, core carriers) using a variety of techniques known in the art. Carrier particles are selected from materials which have a suitable density in the range of particle sizes typically used for intracellular delivery from an appropriate particle-mediated delivery device. The optimum carrier particle size will, of course, depend on the diameter of the target cells. Alternatively, colloidal gold particles can be used wherein the coated colloidal gold is administered (for example, injected) into tissue (for example, skin or muscle) and subsequently taken-up by immune-competent cells.

Tungsten, gold, platinum and iridium carrier particles can be used. Tungsten and gold particles are preferred. Tungsten particles are readily available in average sizes of 0.5 to 2.0 μm in diameter. Although such particles have optimal density for use in particle acceleration delivery methods, and allow highly efficient coating with DNA, tungsten may potentially be toxic to certain cell types. Gold particles or microcrystalline gold (for example, gold powder A1570, available from Engelhard Corp., East Newark, N.J.) will also find use with the present methods. Gold particles provide uniformity in size (available from Alpha Chemicals in particle sizes of 1-3 μm, or available from Degussa, South Plainfield, N.J. in a range of particle sizes including 0.95 μm) and reduced toxicity.

A number of methods are known and have been described for coating or precipitating DNA or RNA onto gold or tungsten particles. Most such methods generally combine a predetermined amount of gold or tungsten with plasmid DNA, CaCl₂ and spermidine. The resulting solution is vortexed continually during the coating procedure to ensure uniformity of the reaction mixture. After precipitation of the nucleic acid, the coated particles can be transferred to suitable membranes and allowed to dry prior to use, coated onto surfaces of a sample module or cassette, or loaded into a delivery cassette for use in a suitable particle delivery instrument, such as a gene gun. Alternatively, nucleic acid vaccines can be administered via a mucosal membrane or through the skin, for example, using a transdermal patch. Such patches can include wetting agents, chemical agents and other components that breach the integrity of the skin allowing passage of the nucleic acid into cells of the subject.

Therapeutic compositions that include a disclosed therapeutic agent can be delivered by way of a pump (see Langer, supra; Sefton, CRC Crit. Ref Biomed. Eng. 14:201, 1987; Buchwald et al., Surgery 88:507, 1980; Saudek et al., N. Engl. J. Med. 321:574, 1989) or by continuous subcutaneous infusions, for example, using a mini-pump. An intravenous bag solution can also be employed. One factor in selecting an appropriate dose is the result obtained, as measured by the methods disclosed here, as are deemed appropriate by the practitioner. Other controlled release systems are discussed in Langer (Science 249:1527-33, 1990).

In one example, a pump is implanted (for example see U.S. Pat. Nos. 6,436,091; 5,939,380; and 5,993,414). Implantable drug infusion devices are used to provide patients with a constant and long-term dosage or infusion of a therapeutic agent. Such device can be categorized as either active or passive.

Active drug or programmable infusion devices feature a pump or a metering system to deliver the agent into the patient's system. An example of such an active infusion device currently available is the Medtronic SYNCHROMED™ programmable pump. Passive infusion devices, in contrast, do not feature a pump, but rather rely upon a pressurized drug reservoir to deliver the agent of interest. An example of such a device includes the Medtronic ISOMED™.

In particular examples, therapeutic compositions including a disclosed therapeutic agent are administered by sustained-release systems. Suitable examples of sustained-release systems include suitable polymeric materials (such as, semi-permeable polymer matrices in the form of shaped articles, for example films, or microcapsules), suitable hydrophobic materials (for example as an emulsion in an acceptable oil) or ion exchange resins, and sparingly soluble derivatives (such as, for example, a sparingly soluble salt). Sustained-release compositions can be administered orally, parenterally, intracistemally, intraperitoneally, topically (as by powders, ointments, gels, drops or transdermal patch), or as an oral or nasal spray. Sustained-release matrices include polylactides (U.S. Pat. No. 3,773,919, EP 58,481), copolymers of L-glutamic acid and gamma-ethyl-L-glutamate (Sidman et al., Biopolymers 22:547-556, 1983, poly(2-hydroxyethyl methacrylate)); (Langer et al., J. Biomed. Mater. Res. 15:167-277, 1981; Langer, Chem. Tech. 12:98-105, 1982, ethylene vinyl acetate (Langer et al., Id.) or poly-D-(−)-3-hydroxybutyric acid (EP 133,988).

Polymers can be used for ion-controlled release. Various degradable and nondegradable polymeric matrices for use in controlled drug delivery are known in the art (Langer, Accounts Chem. Res. 26:537, 1993). For example, the block copolymer, polaxamer 407 exists as a viscous yet mobile liquid at low temperatures but forms a semisolid gel at body temperature. It has shown to be an effective vehicle for formulation and sustained delivery of recombinant interleukin-2 and urease (Johnston et al., Pharm. Res. 9:425, 1992; and Pec, J. Parent. Sci. Tech. 44(2):58, 1990). Alternatively, hydroxyapatite has been used as a microcarrier for controlled release of proteins (Ijntema et al., Int. J. Pharm. 112:215, 1994). In yet another aspect, liposomes are used for controlled release as well as drug targeting of the lipid-capsulated drug (Betageri et al., Liposome Drug Delivery Systems, Technomic Publishing Co., Inc., Lancaster, Pa., 1993). Numerous additional systems for controlled delivery of therapeutic proteins are known (for example, U.S. Pat. No. 5,055,303; U.S. Pat. No. 5,188,837; U.S. Pat. No. 4,235,871; U.S. Pat. No. 4,501,728; U.S. Pat. No. 4,837,028; U.S. Pat. No. 4,957,735; and U.S. Pat. No. 5,019,369; U.S. Pat. No. 5,055,303; U.S. Pat. No. 5,514,670; U.S. Pat. No. 5,413,797; U.S. Pat. No. 5,268,164; U.S. Pat. No. 5,004,697; U.S. Pat. No. 4,902,505; U.S. Pat. No. 5,506,206; U.S. Pat. No. 5,271,961; U.S. Pat. No. 5,254,342; and U.S. Pat. No. 5,534,496).

It may be advantageous to include one or more additional adenovirus vectors in the disclosed compositions. The additional adenovirus vector can include an adenovirus vector that includes a nucleic acid sequence that encodes at least one viral antigen, such as an internal protein, an external protein, or a combination thereof. In some examples an antigen is a viral antigen, such as those discussed above. In some examples, the at least one viral antigen can be an influenza antigen, such as an HA antigen an NA antigen, or a combination thereof. Methods of producing adenovirus vectors and adenoviruses containing influenza antigens can be found in International Patent Application No. PCT/US2006/013384, and those methods are incorporated by reference herein in their entirety.

The additional adenovirus vector can be a human adenovirus vector or a non-human adenovirus vector, such as a porcine adenovirus vector, a bovine adenovirus vector, a canine adenovirus vector, a murine adenovirus vector, an ovine adenovirus vector, an avian adenovirus vector or a simian adenovirus vector. In some examples, the additional adenovirus vector can be a replication defective adenovirus vector made for example by mutation in and/or deletion of at least one of an E1 region gene and an E3 region gene.

### D. Methods of Treatment

This disclosure relates to methods for inhibiting a viral infection in a subject in a subject are disclosed. These methods include selecting a subject in whom the viral infection is to be inhibited and administering an effective amount of the disclosed polypeptides, nucleic acids, adenovirus vectors and/or adenoviruses to a subject, thereby inhibiting the viral infection in the subject. In some embodiments, the viral infection is an infection from a RNA virus, for example a dsRNA virus or a ssRNA virus. In some embodiments, the ssRNA virus is a positive sense ssRNA virus. In other embodiments, the ssRNA virus is a negative sense RNA virus. In some embodiments the ssRNA viral infection is an influenza infection, such as an infection from influenza A, influenza B, a pandemic strain and/or avian strain of influenza. In specific examples, the influenza infection is an infection with influenza strain H5N1, strain H7N7, or strain H9N2.

In some embodiments, a subject who already has a viral infection is selected for administration of an effective amount of the disclosed adenovirus vectors. In other embodiments, a subject who does not yet have a viral infection is selected for administration of an effective amount of the disclosed adenovirus vectors and/or the disclosed adenoviruses. For example, the subject has been exposed to a virus that may result in a viral infection in the subject.

The disclosed polypeptides, nucleic acids and adenovirus vectors are particularly useful in enhancing the effectiveness of a viral vaccine, for example by enhancing immunogenic response to an antigen. Thus a subject may be selected in whom the effectiveness of a viral vaccine is desirable. Disclosed herein are methods for enhancing a viral vaccine's effectiveness in a subject, for example the effectiveness of an RNA viral vaccine, such as a dsRNA viral vaccine or a ssRNA viral vaccine. These methods include administering the disclosed adenovirus vectors to a subject in conjunction with a viral vaccine, thereby enhancing the effectiveness of the vaccine. It is contemplated that the disclosed adenovirus vectors and/or the disclosed adenoviruses can be administered prior to, concurrent with, or after administering a viral vaccine. In some embodiments the viral vaccine is a vaccine for an RNA virus, such as a dsRNA virus or a ssRNA virus. In some examples, the ssRNA viral vaccine is an influenza vaccine, such as a vaccine against influenza A, influenza B, one or more avian or pandemic strains of influenza, for example influenza strain H5N1, strain H7N7, strain H9N2, or a combination thereof.

In some embodiments, the viral vaccine is an adenovirus vector that contains a nucleic acid sequence that encodes at least one viral antigen. In some embodiments, the viral antigen is an internal protein or an external protein. For example an antigen can be a polypeptide expressed on the surface of a virus, such as a viral envelope protein. Examples of antigens include antigens selected from animal and human viral pathogens as described above. Flt3 ligand has been shown to expand the population of dendritic cells. Thus it can also be advantageous to administer Flt3 ligand or a nucleic acid encoding Flt3 ligand to a subject.

### EXAMPLES

### Example 1

### In Vitro Culture of Virus and Cell Lines and Construction of Plasmids

This example describes the conditions used to culture the indicated viruses and cell lines as well as general procedures used in the examples.

Cell lines and viruses: A549 and 293T cells were grown in DMEM (Life Technologies, Grand Island, N.Y.) supplemented with 10% fetal bovine serum (HyClone Laboratories, Logan, Utah), 100 U/ml penicillin and 100 μg/ml streptomycin. Influenza viruses A/Puerto Rico/8/34 (PR8; H1N1) and A/Panama/2007/99 (H3N2) were grown in 10-day-old embryonated hen's eggs at 33.5° C. for 48 hr, while a highly pathogenic avian influenza (HPAI) virus A/Vietnam/1203/2004 (H5N1) was grown in eggs at 37° C. for 24 hours. All trials with HPAI virus were performed in a biosafety level 3 laboratory with enhancement. Unless specified, infection of cells by virus was performed at a multiplicity of infection (MOI) of 1 plaque forming unit (P.F.U.) per cell in a 6-well plate without trypsin supplementation. Influenza viruses were quantified by plaque assay on MDCK cells.

Plasmids and small interfering RNA (siRNA): The pCAGGS-myc-NS1 was constructed by cloning a full-length cDNA of segment 8 from influenza PR8 virus into expression vector pCAGGS with a fusion sequence encoding c-myc-tag located at the 5′ end of cloned cDNA. The splice acceptor sequence was mutated by overlap PCR. Constructs that express domains of NS1, pCAGGS-myc-NS1aa1-80 and pCAGGS-myc-NS1aa81-230, were derived from pCAGGS-myc-NS1. The pEF-FLAG-RIG-I, pEF-FLAG-N-RIG-I, and pEF-FLAG-C-RIG-I plasmids have been described (Yoneyama et al., Nat. Immunol. 5:730-737, 2004). The (-110-IFNβ-CAT, (PRDIII-I)₃-CAT, pEF-Bos-TRIF, and pcDNA3-IKKε have also been described. The pUNO-hIPS1 was obtained from INVIVOGEN™ (San Diego, Calif.). Predesigned siRNA targeting human RIG-I (siRIG-I), human MDA5 (siMDA5) and control siRNA targeting luciferase (siLuc) were purchased from Dharmacon (Chicago, Ill.).

Real Time RT-PCR: Real time RT-PCR was performed as described previously (Guo Z et al., J. Immunol. 175:7407-7418, 2005). Two sets of PCR assays were performed for each sample using primers specific for cDNA of the following genes: RIG-I, IFNβ, TNF-α, ISG15, MxA, and GAPDH. PCR product from the above genes was cloned into PCR-Blunt II-TOPO vector (INVIVOGEN™, Carlsbad, Calif.) and the cloned constructs were used to create standard curves in real time PCR. The cycle threshold of each sample was converted to copy number of cDNA per μg of RNA and was normalized to GAPDH quantity of the corresponding sample. Unless specified, all assays were performed at least three times from independent RNA preparations.

Transient transfection: Transient transfections of plasmid were carried out using FuGENE 6 transfection reagent from Roche (Indianapolis, Ind.) according to the manufacturer's protocols. For transient transfection of dsRNA into 293T cells, 0.2 μg of poly (I:C) (Sigma-Aldrich) was transfected with LIPOFECTAMINE™ 2000 (INVIVOGEN™). Transient transfections of siRNA into A549 cells were conducted using DharmaFECT 1 (Dharmacon) according to the manufacturer's protocols.

Western blot: Western blot was performed as described previously (Guo Z et al., J. Immunol. 175:7407-7418, 2005). Antibodies against FLAG-tag and β-actin were purchased from Sigma-Aldrich, and c-myc-tag from Invitrogen. Antibody against human RIG-I was purchased from IBL (Gunma, Japan). Antibody against human MDA5 was described previously (Yoneyama et al., J. Immunol. 175:2851-2858, 2005).

### Example 2

### RIG-I Mediated IFNβ Response to IAV Infection in Lung Epithelial Cells

This example demonstrates RIG-I mediation of the induction of IFNβ production in response to influenza A viral infection of human lung epithelial cells.

To determine whether RIG-I is needed for IFN-I response to IAV infection, endogenous expression of RIG-I in the human lung epithelial cell line A549 was knocked down using RNA interference (RNAi) using predesigned siRNA targeting human RIG-I (siRIG-I) purchased from Dharmacon (Chicago, Ill.). Control siRNA targeting luciferase (siLuc) was purchased from Dharmacon (Chicago, Ill.). Endogenous expression of RIG-I in the human lung epithelial cell line A549 was knocked down using RNA interference (RNAi), by transient transfection using DharmaFECT 1 (Dharmacon) according to the manufacturer's protocols.

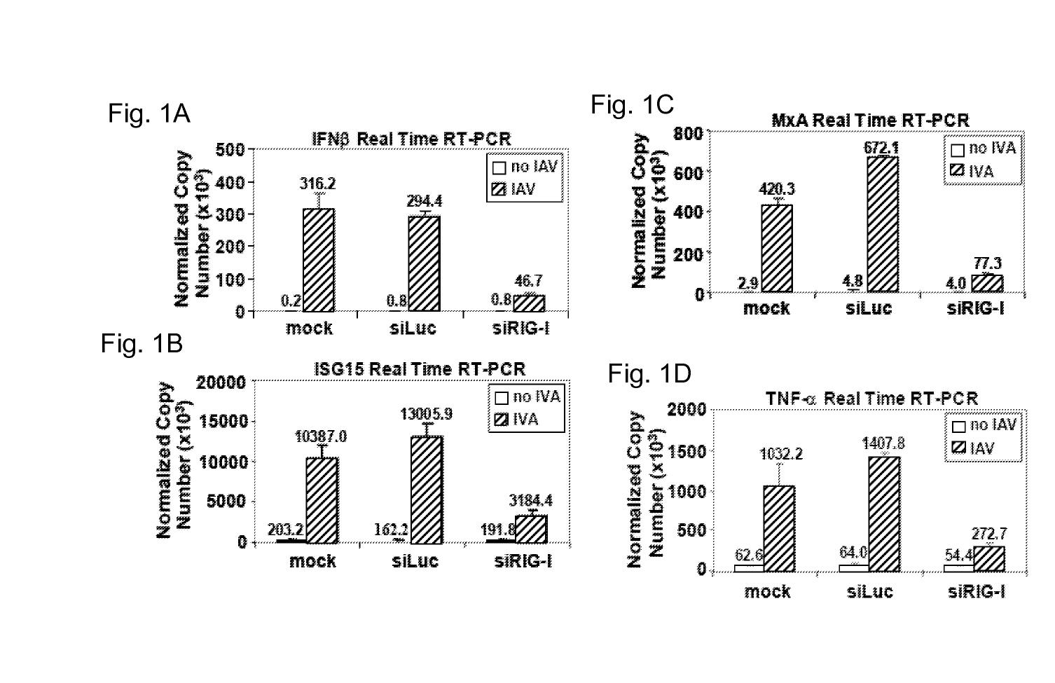

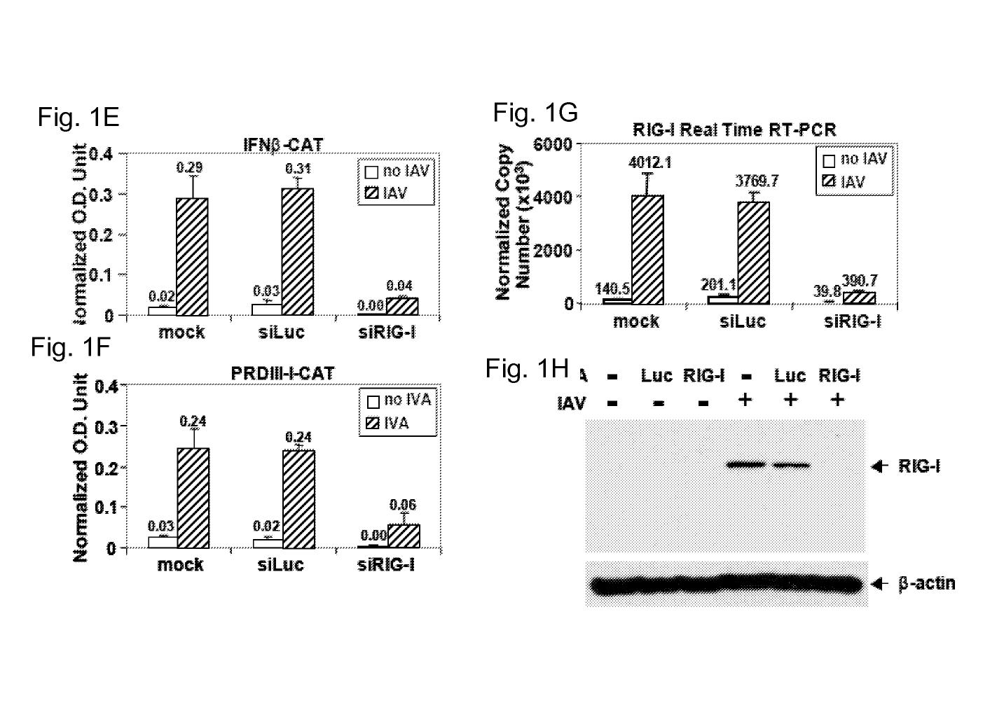

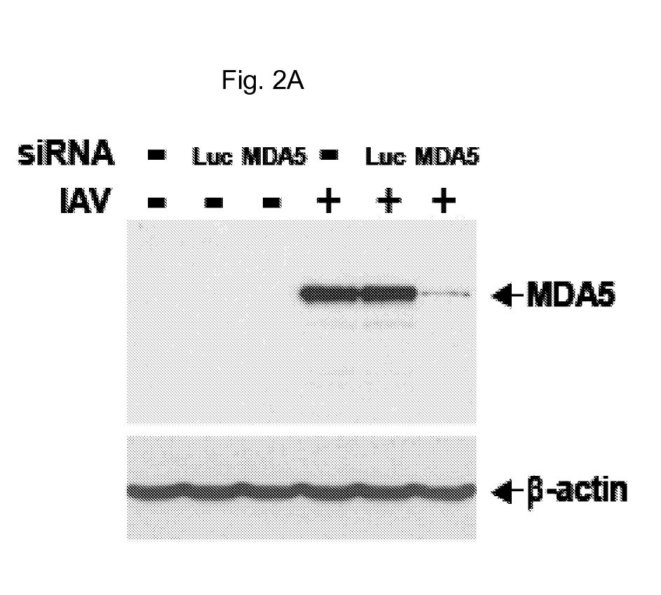

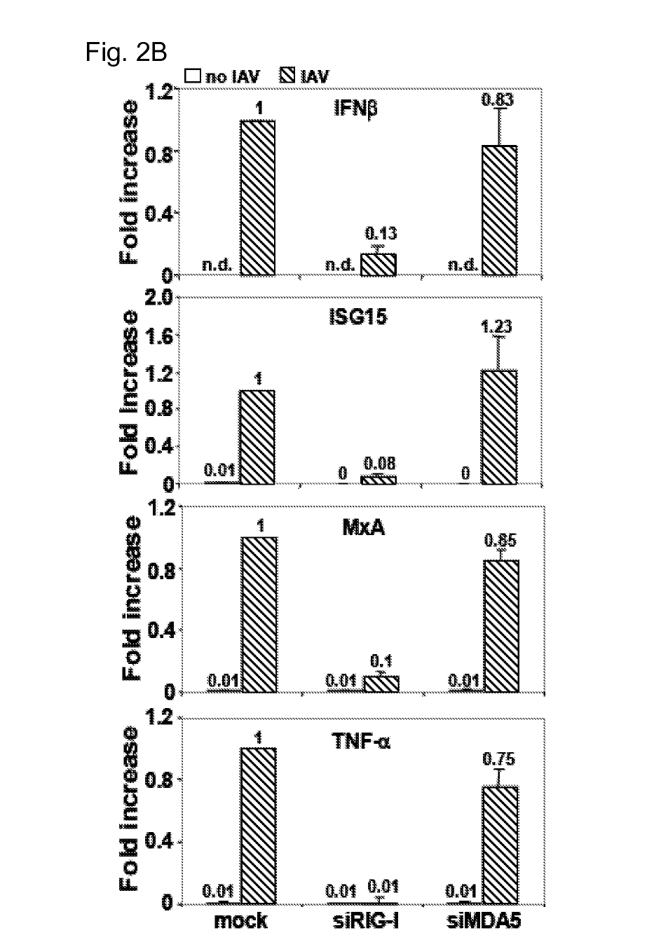

The cells were incubated for 24 hours following introduction of the siRNA and then infected with influenza virus A/Panama/2007/99 (H3N2). Transfection of small interfering RNA (siRNA) targeting RIG-I, but not a control siRNA targeting the luciferase gene, greatly reduced the level of IFNβ mRNA induced 16 hours post infection with IAV. This result demonstrates the pivotal role for RIG-I in IFN-I response to IAV infection in human lung epithelial cells (FIG. 1A). Similarly, the induction of type I IFN-inducible genes, ISG15 and MxA were greatly reduced in cells transfected with siRNA targeting RIG-I (FIGS. 1B & C). It has been shown that the RIG-I signaling pathway bifurcates to activate IRF-3 and NF-κB (Yoneyama et al., Nat. Immunol. 5:730-737, 2004). To determine whether RIG-I plays a role in IAV-induced expression of NF-κB-responsive genes, the expression level of TNF-α was analyzed (Collart et al., Mol. Cell. Biol. 10:1498-1506, 1990), in RIG-I knocked-down cells (FIG. 1D). The induction level of TNF-α was also greatly reduced in cells transfected with siRNA targeting RIG-I, indicating that the signaling pathway leading to NF-κB activation by IAV infection might require RIG-I function. The importance of RIG-I in the IFN-I response to IAV infection was also demonstrated by IFNβ promoter and IRF3-responsive promoter reporter assays. Consistent with the results from real time RT-PCR, IFNβ promoter [IFNβ-CAT] (FIG. 1E) or IRF-3-responsive promoter [PRDIII-1-CAT] (FIG. 1F) reporter expression was decreased in RIG-I knocked-down cells as compared to controls. The specificity of RNAi was evidenced by the greatly reduced expression of RIG-I mRNA and protein only in cells transfected with siRNA targeting RIG-I (FIGS. 1G & H). Taken together, these data indicate that RIG-I is essential for induction of IFN-I and TNF-α in response to IAV infection, and that the induction activity involves activation of IRF-3 and NF-κB. Melanoma differentiation associated gene 5 (MDA5), an RNA helicase related to RIG-I, has been shown to share a common signaling cascade with RIG-I (Yoneyama et al., J. Immunol. 175:2851-2858, 2005). To determine whether MDA5 plays a role similar to RIG-I in IFN-I response to IAV infection, endogenous expression of MDA5 in A549 cells was knocked down by RNAi, and the cells infected with IAV 24 hours later. As expected, the expression of MDA5 was induced by IAV infection and this induction was greatly reduced only in cells transfected with siRNA targeting MDA5 (FIG. 2A). However, in comparison to RIG-I, transfection of siRNA targeting MDA5 only marginally reduced the level of expression of IFNβ, ISG15, MxA, and TNF-α induced by IAV infection (FIG. 2B), indicating that MDA5 is not essential for IFN-I response to IAV infection in this human lung epithelial cell line.

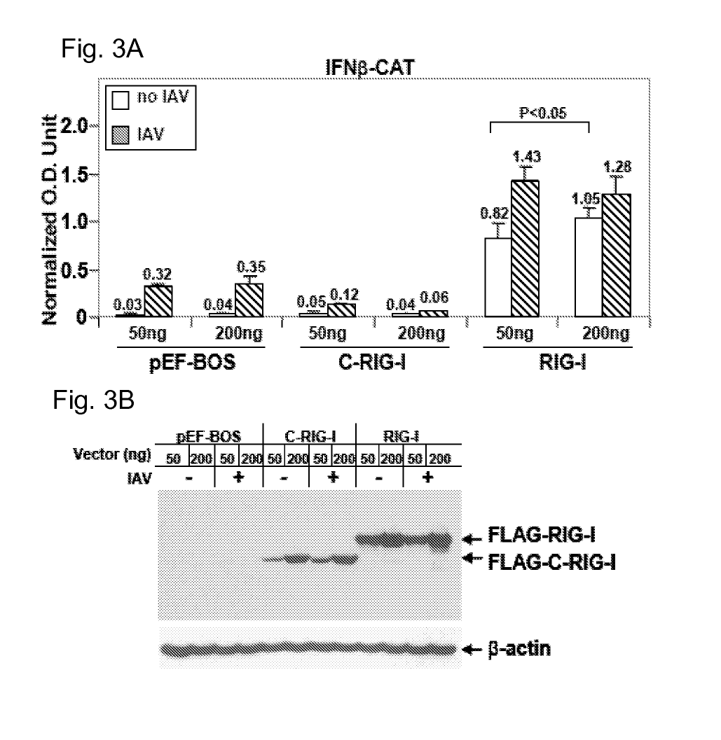

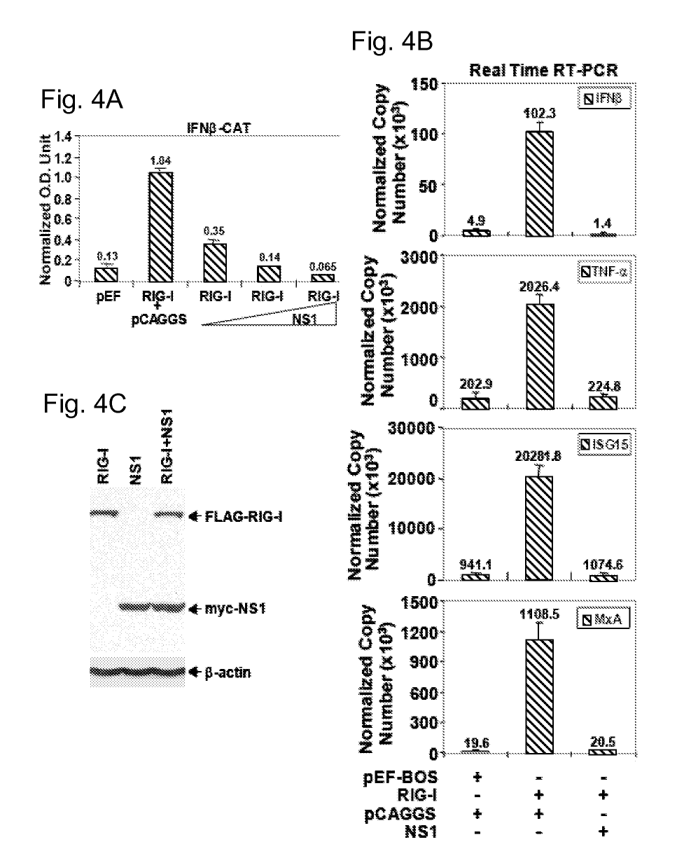

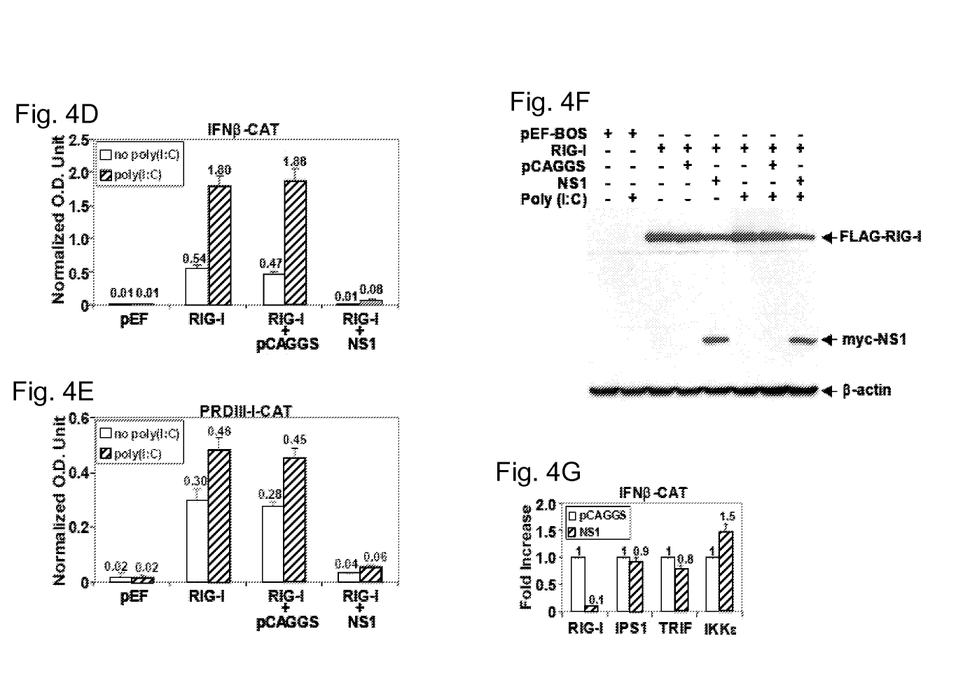

An alternative approach to demonstrate the critical role of RIG-I in the IFN-I response to IAV infection relied on transient over-expression of FLAG-tagged RIG-I (FIG. 3A). Transient transfection of a full-length RIG-I expression vector into 293T cells was sufficient to induce CAT expression from the IFNβ-CAT reporter in a dose-dependent manner. IAV infection further enhanced the level of induction, which might occur through enhanced expression of endogenous RIG-I after IAV infection. Similarly, endogenous expression of IFNβ, ISG15, MxA and TNF-α, (FIG. 4B) was induced by transient over-expression of RIG-I in A549 cells and their expression was also further induced by IAV infection.

### Example 3

### Expression of C-RIG-I can Block IAV-Initiated IFNβ Induction

This example describes the determination of the ability of the polypeptides containing the C-terminal helicase domain of RIG-I to block IAV-initiated IFNβ induction.

To determine whether expression of C-RIG-I can block IAV-initiated IFNβ induction, 293T cells were co-transfected with a FLAG-tagged C-RIG-I expression vector and the IFNβ-CAT reporter construct, and infected with IAV 24 hours later. The induction level of IFNβ reporter was inhibited by C-RIG-I in a dose-dependent manner (FIG. 3A), confirming that C-RIG-I is a dominant negative inhibitor for IFNβ induction by IAV infection and RIG-I does play an important role in IFN-I response to IAV infection. The ectopic expression of RIG-I and C-RIG-I was confirmed by western blot analysis (FIG. 3B).

### Example 4

### Inhibition of RIG-I Induction of Type I Interferon by Nonstructural Protein One of Influenza A

This example describes the inhibition of RIG-1-initiated induction of type I IFN by influenza A virus (IAV) nonstructural protein one (NS1).

Influenza virus lacking the NS1 gene is a potent inducer of IFN-I and NS1 has been shown to inhibit activation of IRF-3 (Basler et al., J. Virol. 77:7945-7956, 2003). However, the precise mechanism by which NS1 antagonizes induction of IFN-I remains unknown. The critical role of RIG-I in the IFNβ response to IAV infection prompted the hypothesis that NS1 targets the RIG-I signaling pathway and inhibits production of IFN-I. To demonstrate this effect, RIG-I expression construct and IFNβ-CAT reporter were co-transfected with various amounts of NS1 expression vector into A549 cells, and the activity of IFNβ promoter was analyzed by CAT ELISA. Transfection of the RIG-I expression vector alone greatly induced CAT expression from the IFNβ-CAT reporter, and co-transfection of the NS1 expression vector inhibited the induction activity of RIG-I in a dose-dependent manner (FIG. 4A). Similarly, the endogenous expression of IFNβ, ISG15, MxA, and TNF-α was greatly induced by overexpression of RIG-I, and co-transfection of the NS1 expression vector almost completely blocked the induction (FIG. 4B). It should be noted that transfection of NS1 expression vector alone caused a slight reduction (less than 2-fold) in the basal level of IFNβ expression. However, the inhibitory function of NS1 on RIG-I signaling was not due to altered expression of RIG-I, as comparable levels of RIG-I expression were found in cells transfected with RIG-I or RIG-I plus NS1 expression constructs (FIG. 4C).

Next, it was determined whether NS1 could inhibit RIG-I activity in the presence of dsRNA. RIG-I expression vector and IFNβ promoter reporter plasmids were transfected with or without the NS1 expression vector into 293T cells. After 24 hours of incubation, cells were transfected with dsRNA (poly (I:C)) and incubated for 8 hours to induce IFN-I. The activity of IFNβ promoter was determined by CAT ELISA. Transfection of the RIG-I expression vector induced CAT expression driven by the IFNβ promoter, and the level of induction was further increased in cells transfected with poly (I:C), indicating that interaction of RIG-I with dsRNA enhanced the signaling activity of RIG-I (FIG. 4D). Most importantly, the induction function of RIG-I was greatly inhibited by NS1 in the presence or absence of poly (I:C). CAT expression driven by IRF-3-responsive promoter was also downregulated by co-expression of NS1 (FIG. 4E). Comparable levels of RIG-I expression were found in cells transfected with RIG-I or RIG-I plus NS1 expression constructs (FIG. 4F). In addition, co-transfection of NS1 with IPS1, TRIF, or IKKε expression vectors failed to inhibit production of IFN-I that was induced by overexpression of these molecules, indicating the specificity of NS1 inhibitory activity on the RIG-I pathway (FIG. 4G).

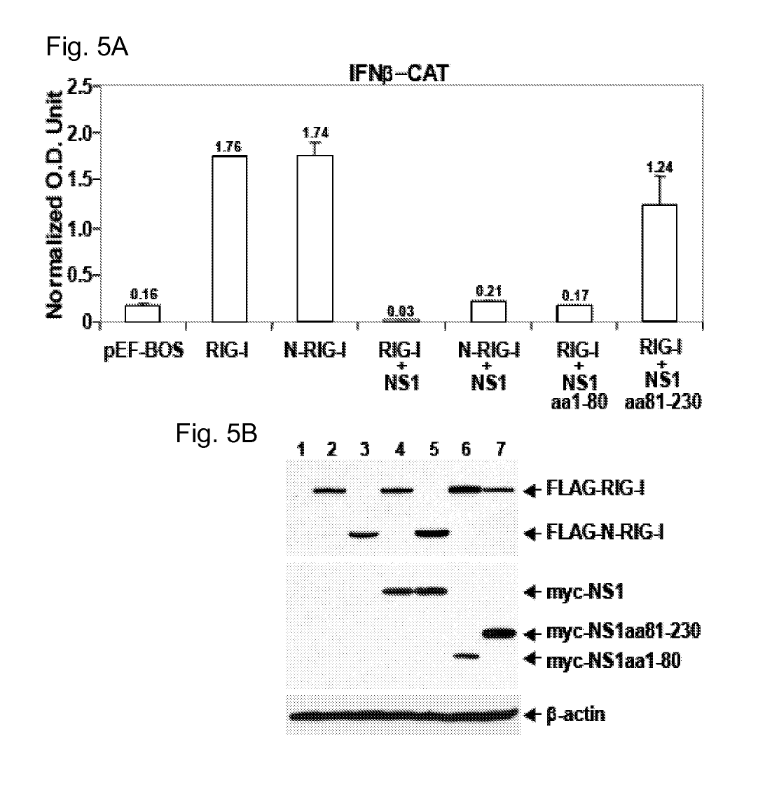

To further determine the interaction between RIG-I and NS1, constructs that expressed domains of RIG-I or NS1 and IFNβ-CAT reporter plasmids were transfected with or without the full-length NS1 or RIG-I expression vectors into A549 cells (FIG. 5A). Transfection of the N-RIG-I expression vector greatly induced CAT expression from the IFNβ promoter reporter, and co-transfection of the NS1 expression vector inhibited the induction activity of N-RIG-I. Additionally, co-transfection of the constructs that expressed the N-terminus (amino acids 1-80), but not the C-terminus (amino acids 81-230) of NS1 with the RIG-I expression vector greatly repressed the induction of IFNβ-CAT reporter. Comparable levels of RIG-I expression were found in cells transfected with RIG-I or RIG-I plus NS1-domain expression vectors (FIG. 5B).

NS1 of IAV is a multifunctional viral protein (Krug et al., Virology 309:181-189, 2003). Two cellular proteins that are required for the 3′-end processing of cellular pre-mRNAs, the 30-kDa subunit of the cleavage and polyadenylation specificity factor (CPSF) and poly (A)-binding protein II (PABII), are bound and inactivated by IAV NS1, leading to decreased expression of the early type I IFN-independent antiviral genes (Krug et al., Virology 309:181-189, 2003). NS1 also inhibits the activation of another cellular antiviral gene, protein kinase R (PKR). Activation of PKR is known to phosphorylate the α-subunit of the translation initiation factor eIF2 to inhibit protein synthesis and therefore virus replication (Krug et al., Virology 309:181-189, 2003). This result presents further evidence that NS1 antagonizes the host antiviral response by targeting and inhibiting RIG-I signaling to block IRF-3 activation. It should be noted that NS1 inhibits the activity of RIG-I in the presence and absence of poly (I:C). The anti-IFN properties of IAV NS1 have been mapped to its N-terminal dsRNA-binding domain (Wang et al., J. Virol. 74:11566-11573, 2000). This data is consistent with the observation and indicates that the N-terminal domain of NS1 is sufficient to counteract RIG-I activity (FIG. 5A).

### Example 5

### RIG-I Inhibits IAV Replication

This example describes the procedures for demonstrating that ectopic expression of RIG-I inhibits the replication of influenza A virus in vivo.

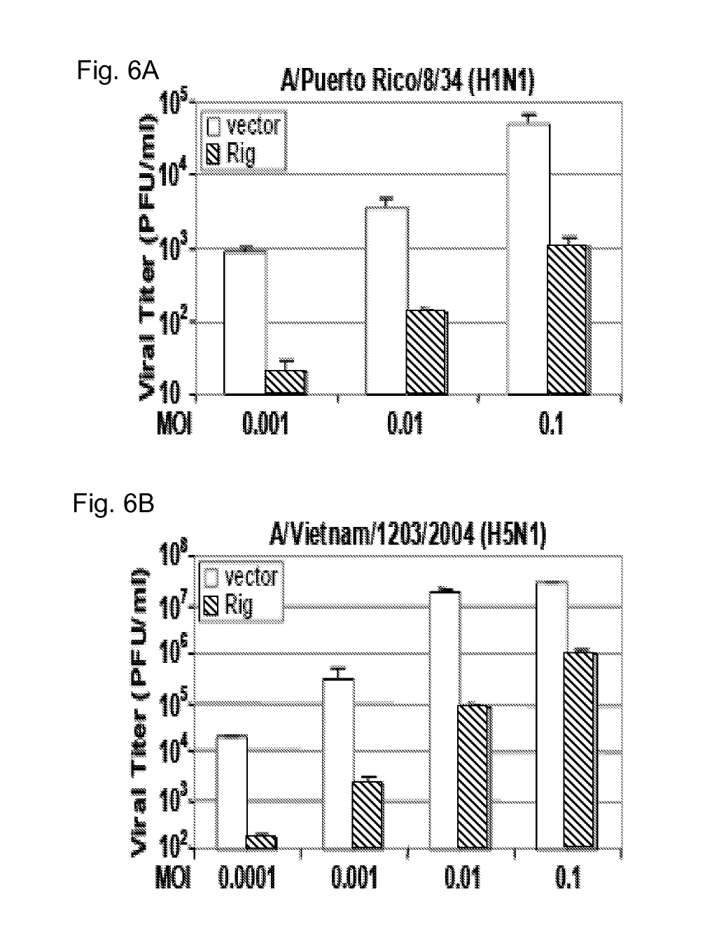

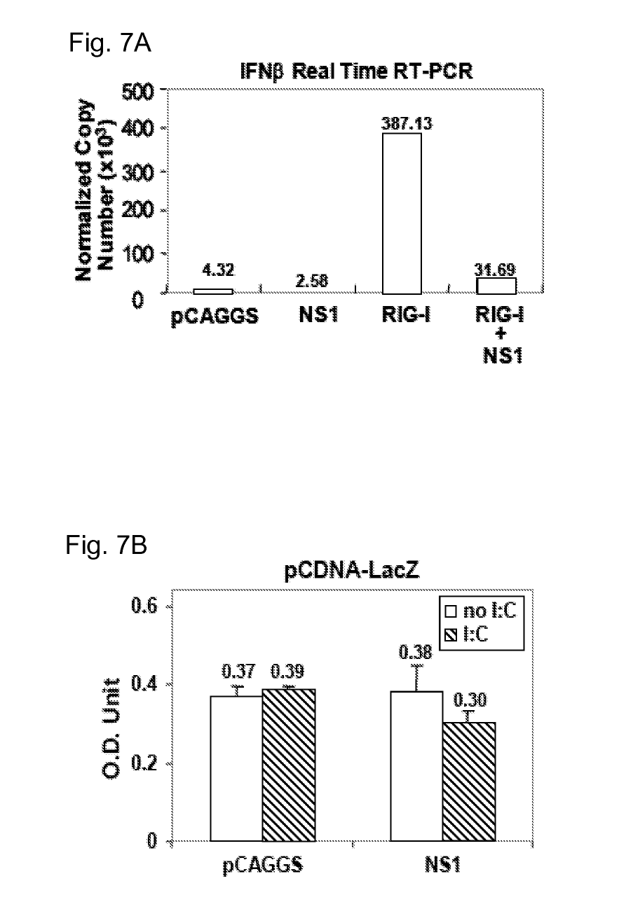

Increased expression of RIG-I has been shown to reduce the yield of vesicular stomatitis virus and encephalomyocarditis virus (Yoneyama et al., Nat. Immunol. 5:730-737, 2004). To test whether RIG-I can inhibit replication of influenza virus, A549 cells were transiently transfected with the construct that expressed full-length RIG-I or its null expression control vector, and 24 hours later were infected with IAV PR8 or highly pathogenic avian influenza virus A/Vietnam/1203/2004 (H5N1) at various MOI in the absence of trypsin. Compared to cells transfected with control vector, the yields for PR8 and H5N1 virus were reduced by 1 to 2 log of control in cells transfected with RIG-I expression vector (FIGS. 6A & B). This result demonstrates inhibition of H1N1 and H5N1 IAV replication by RIG-I and the general capacity of RIG-I in anti-influenza function.

### Example 6

### Immunogenicity of Adenoviral(Ad)-Vector Mediated Delivery of RIG-I

This example describes the induction of IFN in a subject by adenoviral(Ad)-vector mediated delivery of RIG-I.

To determine the optimal dose for the induction of IFN, BALB/c mice (3-4 month old naïve or previously primed with a human H1N1 virus) are immunized by intranasal (i.n.) route with 1×10^{8,} 5×10⁷, 1×10⁷, 5×10⁶, 1×10⁶ and 5×10⁵ p.f.u. of Ad-vector expressing N-terminal RIG-I and Ad-vector expressing H5HA with or without M2 & NP. Negative controls include animals that were immunized with Ad-vector alone. IFN-levels in lung tissue are determined by ELISA at 24 hour intervals. Similarly, the expression of HA, M2, and NP is determined by ELISA. Based on the results of these studies the optimal dose and time to deliver H5HA with or without M2 & NP following the induction of IFN is determined.

### Example 7

### Immunogenicity of Adenoviral (Ad)-Vector Mediated Delivery of RIG-I and Flt-3L

This example describes the immunogenicity of adenoviral(Ad)-vector mediated delivery of RIG-I, Flt-3L, and H5 HA from A/Indonesia/5/05 with or without M2 and NP.

To determine the optimal dose for the induction of IFN and mobilization of DCs, the young BALB/c mice (3-4 month old naïve or previously primed with a human H1N1 virus) are immunized by intranasal (i.n.) route with 1×10^{8,} 5×10⁷, 1×10⁷, 5×10⁶, 1×10⁶ and 5×10⁵ p.f.u. of Ad-vector expressing N-terminal RIG-I and Flt-3L and Ad-vector expressing H5HA with or without M2 & NP. Negative controls include animals that are immunized with Ad-vector alone. IFN-levels in lungs and the frequency of DCs in lungs, mediastinal lymph nodes are determined by ELISA and flow cytometry with various activation and DC-specific markers at 24 hour intervals. Similarly, the expression of HA, M2, and NP is determined by ELISA. Based on the results of these studies the optimal dose and time to deliver H5HA with or without M2 & NP following the induction of IFN and mobilization of DCs can be determined.

### Example 8

### Cell-Mediated Immune Responses Following the Delivery of H5HA with or without M2 & NP

This example describes the determination of serological and cell-mediated immune responses following the delivery of H5HA with or without M2 & NP

3-4 month old young Balb/c mice of (naïve or previously primed with a human H1N1 virus) are immunized with H5HA with or without M2 and NP following the induction of IFN and DC mobilization. The animals receive one or two immunizations at 4 wk intervals. Sera is collected 3 weeks post-immunization from all mice to monitor the isotype of the H5- and M2-specific antibodies by ELISA, and H5-neutralizing antibody responses by micro-neutralization assay. Since HA 518 [HA₅₁₈₋₅₂₆ (IYSTVASSL; SEQ ID NO:5)] and NP 147 [NP₁₄₇₋₁₅₅ (TYQRTRALV; SEQ ID NO:6)] epitopes conserved in all currently circulating avian and human H5N1 viruses, CD8 T cell responses are determined using epitope-specific pentamers (K^{d} tetramers are unstable), IFN-β secreting cells by ICCS, and by cytotoxicity assay from mediastinal lymph nodes and spleens 2-3 wks post-immunization. HA- and M2-epitope-specific CD4 T cell responses are determined by IL-2 and/or IFN-β ICCS or ELISpots.

### Example 9

### Determination of Protective Immune Responses Against Lethal Challenge

This example describes the procedures used to determine the protective immune responses generated by the immunization schemes of Examples 6-8.

At 4 weeks post-primary or secondary vaccination, all animals are challenged i.n. with homologous (A/Indonesia/5/05) or antigenically distinct strains of H5N1 (A/HK/483/97, A/HK/213/03, and A/VN/1203/04). The lungs are harvested from a cohort of mice/group on day 3 post-challenge to determine viral titers in embryonated chicken eggs. The remaining mice/group are monitored for morbidity and mortality by measuring loss in body weight and survival for 14 days post-challenge.

### Example 10

### Determination of the Immunogenicity and Protection in Aged Subjects

This example describes the immunogenicity and protection of candidate vaccines in aged mice.

Preliminary evidence indicates that IFN levels declines with age, which may be responsible for increased susceptibility of elderly to viral infections and poor adaptive immune responses. Two to three different doses of Ad-vectors expressing N-terminal RIG-I, Ad-vectors expressing N-terminal RIG-I and Flt-3L and Ad-vectors expressing H5HA with or without M2 and NP are chosen. Aged mice (naïve Balb/c mice>24 months old or Balb/c mice that were primed previously with a human H1N1 virus and aged) are immunized with an optimal dose of the vaccine candidate once or twice at 4 wks apart. Humoral and cell-mediated immune responses are assessed. HA- and M2-epitope-specific CD4 T cell responses are determined by IL-2 and/or IFN-γ ICCS or ELISpots.

### Example 11

### Determining the Longevity of Protective Immune Response in Young and Aged Subjects

This example describes the determination of the longevity of protective immune response in young and aged mice

After immunization of naïve and H1N1-primed young animals, sera is collected at 4, 6, 8 and 12 months post-vaccination and determine HA- and M2-specific antibody responses as well as virus neutralization titers. In addition, CD8 and CD4 T cell responses are assessed at each of those times. HA- and M2-epitope-specific CD4 T cell responses are determined by IL-2 and/or IFN-γ ICCS or ELISpots.

### Example 12

### Determining the Therapeutic Activity of a Vaccine Containing N-Terminal RIG-I

This example describes the ability of the vaccine containing N-terminal RIG-I to confer resistance to challenge with homologous and antigenically distinct H5N1 viruses on different days post-immunization before the induction of detectable adaptive immune responses

Since NS1 mediated suppression of IFN responses may be contributing to the observed pathogenicity of H5N1 viruses, the vaccine containing N-terminal RIG-I, which induces IFN without competing for dsRNA with NS1 could be used as a therapeutic vaccine, along with H5HA with or without M2 & NP. Following delivery of N-terminal-RIG-I, Flt-3L and H5HA with or without M2 & NP, groups of animals are challenged on different days (for example, 1 or 2 or 3) and the viral titers are determined on day 3 post-challenge.

### Example 13

### Determining the Therapeutic Activity of a Vaccine Containing N-Terminal RIG-I to Confer Resistance Post-Infection

This example describes the ability of vaccines containing N-terminal RIG-I to confer resistance post-infection to influenza when given post infection.

To assess if this vaccine approach confers protection after the animals are infected, young Balb/c mice are infected with either A/Indonesia/5/05 or antigenically distinct H5N1 viruses. The vaccine candidate is administered once on different days post-infection (day 0, 1, 2, 3, 4, 5, 6, 7, or 8) to groups of mice and the changes in body weight will be determined as a measure of morbidity. Lungs from groups of mice are collected on 3 days post-administration of the vaccine to determine viral titers. This vaccine will have potential therapeutic utility until day 4 or 5 of infection, as majority of the animals succumb to infection

### Example 14

### Creation of Recombinant Adenovirus Vectors Expressing Full Length hRIG-I, C-Terminal hRIG-I, and N-Terminal (CARD Containing) hRIG-I

This example demonstrates the construction of adenoviral vectors containing nucleic acid encoding RIG-I polypeptides.

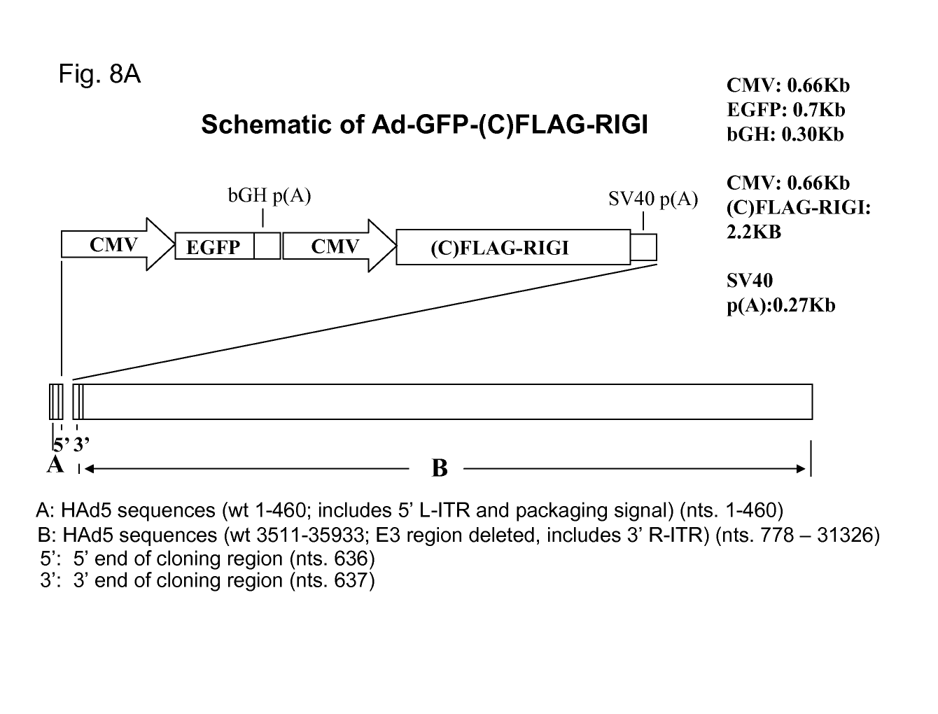

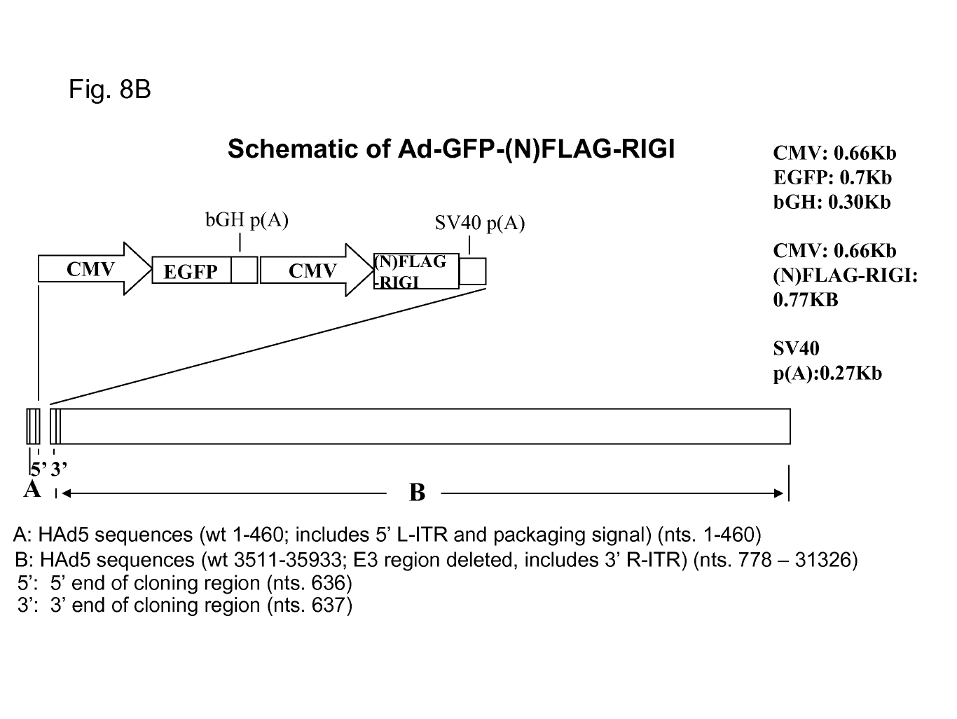

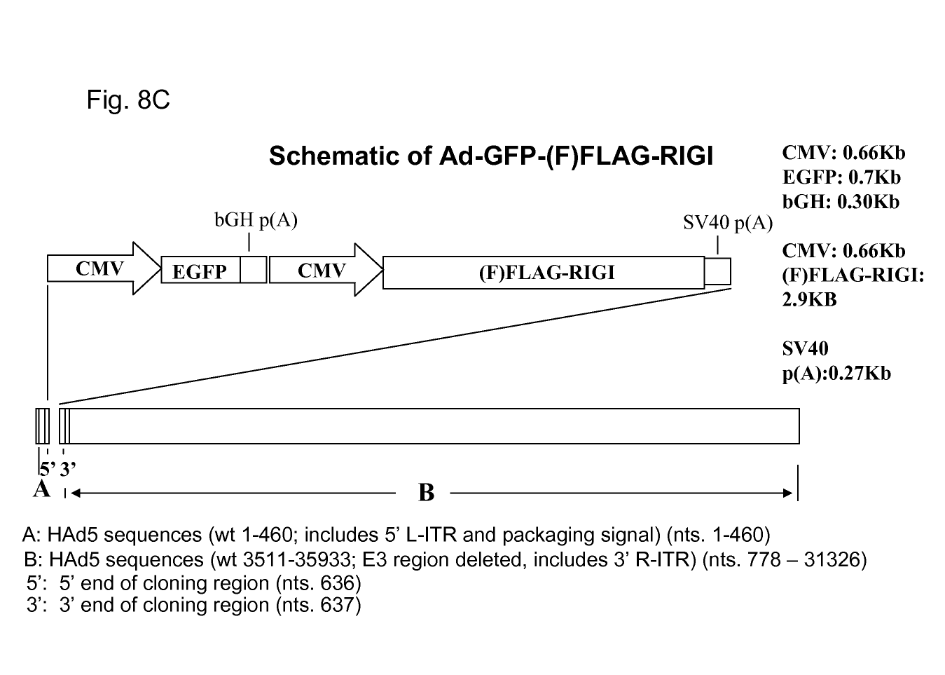

The adenoviral vector constructs shown in FIG. 8A-8C were constructed as follows. Fragments of FLAG tagged C-terminal RIG-I, FLAG tagged N-terminal RIG-I, and full length FLAG tagged RIG-I were obtained from double restriction digests of pEF-FLAG-C-RIG-I, pEF-FLAG-N-RIG-I, and pEF-FLAG-RIG-I, respectively with XbaI and ClaI. The XbaI/ClaI fragments were subcloned into DUAL2GFP-CCM(−) vector through blunt-end ligation. The expression cassette DUAL2GFP-CCM(−) containing the FLAG tagged RIG-I constructs were transferred into the HAd5 viral backbone DNA. The resulting adenoviral vectors (AD-VEC-FLAG-FULL-RIG-I (expressing full length RIG-I protein with an N-terminal FLAG tag), AD-VEC-FLAG-N-TER-RIG-I (expressing the first 228 amino acids of RIG-I with an N-terminal FLAG tag), and AD-VEC-FLAG-C-TER-RIG-I (expressing from amino acid 218 through the stop codon of RIG-I with an N-terminal FLAG tag)) were tested for their ability to infect Human lung epithelial cells (A549) and express RIG-I polypeptides.

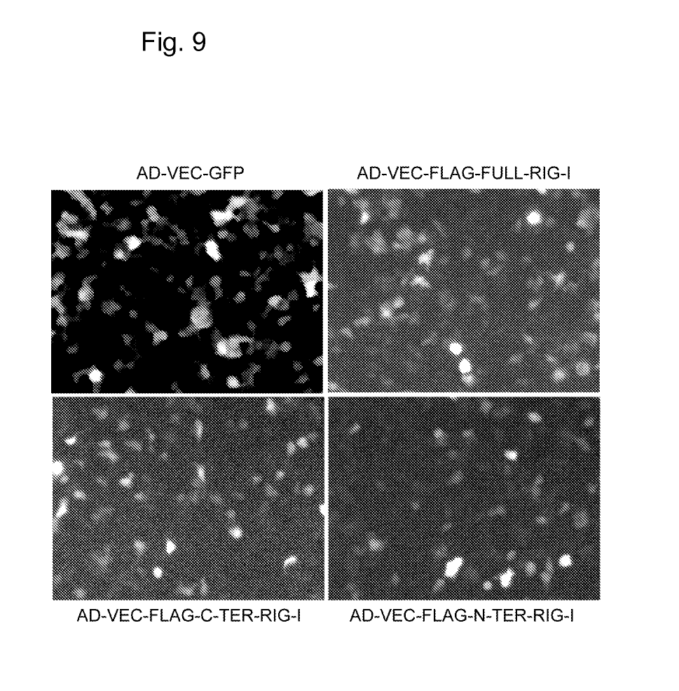

Human lung epithelial cells (A549) in growth medium lacking fetal bovine serum (FBS) were infected at a multiplicity of infection (MOI) of 5 with AD-VEC-GFP (control adenovirus expressing only GFP) and adenoviruses co-expressing GFP and one of three FLAG-tagged RIG-I proteins: AD-VEC-FLAG-FULL-RIG-I (expressing full length RIG-I protein with an N-terminal FLAG tag), AD-VEC-FLAG-N-TER-RIG-I (expressing the first 228 amino acids of RIG-I with an N-terminal FLAG tag), and AD-VEC-FLAG-C-TER-RIG-I (expressing from amino acid 218 through the stop codon of RIG-I with an N-terminal FLAG tag). Digital fluorescent images were captured 72 hours post infection (see FIG. 9). With reference to FIG. 9, the top left panel shows GFP localization in A549 cells infected with AD-VEC-GFP; the top right panel shows GFP localization in A549 cells infected with AD-VEC-FLAG-FULL-RIG-I; and bottom left panel shows GFP localization in A549 cells infected with AD-VEC-FLAG-C-TER-RIG-I; and bottom right panel shows GFP localization in A549 cells infected with AD-VEC-FLAG-N-TER-RIG-I. Over 90% of the cells expressed GFP.

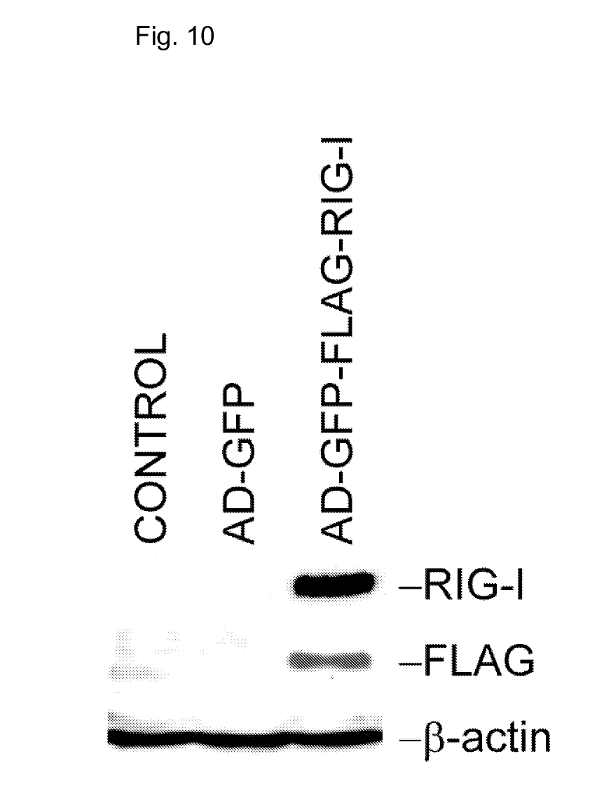

To determine whether the adenoviral vectors expressed RIG-I polypeptide, human lung epithelial cells (A549) were infected at an MOI of 5 with AD-VEC-FLAG-FULL-RIG-I for 72 hours. 72 hours post infection, growth medium was removed and cells were washed twice with PBS. The cells were then lysed in Laemmli buffer containing 5% β-mercaptoethanol, protease inhibitors, subjected to SDS Polyacrylamide Gel Electrophoresis on a 10% gel, and transferred to nitrocellulose membrane for Western blot analysis (see FIG. 10). With reference to FIG. 10, protein lysate from a mock infection (left lane), infection with AD-VEC-GFP (middle lane), and infection with AD-VEC-FLAG-FULL-RIG-I (right lane) were subjected to SDS Polyacrylamide Gel Electrophoresis on a 10% gel and transferred to nitrocellulose membrane. The membrane was then probed with α-RIG-I (top panel), α-FLAG (middle panel), and α-β actin antibodies (bottom panel). As shown in FIG. 10, control A549 or Ad-GFP infected A549 cells did not express RIG-I or FLAG (lane 1 and 2). However, A549 cells infected with Ad-GFP-(full length) FLAG-RIG-I expressed both RIG-I and FLAG as detected by immunoblot (lane 3).

### Example 15

### Creation of Recombinant Adenovirus Vectors Expressing Full Length, CARDs from hRIG-I and MDA5

This example demonstrates the construction of adenoviral vectors containing nucleic acid encoding CARD polypeptides in the absence of a helicase domain, such as RIG-I and MDA5 CARDs in the absence of a helicase domain.

Nucleic acids fragments that encoding residues 1-87 of the amino acid sequence set forth as SEQ ID NO:1, residues 92-172 of the amino acid sequence set forth as SEQ ID NO:1 and residues 1-284 of the amino acid sequence set forth as SEQ ID NO:1 are amplified from the commercially available full length hRIG-I expression vector pUNO hRIG-I, from INVIVOGEN™ using PCR. Nucleic acids fragments that encoding residues 7-97 of the amino acid sequence set forth as SEQ ID NO:3, residues 110-190 of the amino acid sequence set forth as SEQ ID NO:3, and residues 1-196 of the amino acid sequence set forth as SEQ ID NO:3 are amplified from a MDA5 cDNA. The resulting PCR products are then cloned into an entry vector (pENTR D TOPO; Catalog no. 2400-20) which is propagated and maintained in One Shot chemically competent E. coli from INVIVOGEN™ (Catalog no. C7510-03). Using the gateway system from INVIVOGEN™, a LR recombination reaction is performed between the entry plasmid, containing the fragment of interest, and a general destination plasmid, pAd/CMV/V5-DEST (INVIVOGEN™, Catalog no. 493-20). This reaction allows the transfer of the cloned nucleic acid fragment from the entry vector (pENTR D-TOPO) to the destination vector (pAd/CMV/V5-DEST) by site specific recombination. The resulting destination plasmid, containing the fragment of interest, is then selected for using ampicillin and propagated in ONE SHOT® chemically competent E. coli from INVIVOGEN™. This plasmid is then sequenced and verified for the appropriate nucleic acid sequence. Once verified for the proper sequence, each plasmid is purified and digested with the restriction enzyme PacI. After digestion with PacI the linearized plasmid is delivered to 293A cells using the transfection reagent DNA-LIPOFECTAMINE™ 2000 (INVIVOGEN™; Catalog no. 11668-027). 48 hours post-transfection transfected cells are transferred from six well plates to large tissue culture flasks. The cells are then complemented with complete culture media and monitored every 2-3 days for visible regions of cytopathic effect (CPE), typically for a period of 7-10 days. In the meantime media is also replenished as needed. Once approximately 80% CPE is observed (10-13 days post-transfection) the adenovirus containing cells are harvested and crude viral lysate is prepared. From this crude viral lysate recombinant adenovirus is purified (Clonetech Adeno-X purification kit; Catalog no. PT3767-2) and tittered (Clonetech Adeno-X rapid titer kit; Catalog no. PT3767-2). The resulting recombinant adenovirus, containing the desired ORF of hRIG-I, is then further amplified in 293A cells. Crude viral lysate from this second round is then harvested and the recombinant adenovirus is purified and tittered.

### Example 16

### Generation and Characterization of Nonhuman Vectors Expressing Viral Antigens

This example describes the construction of adenoviral vectors containing viral antigens.

Infectious clones containing the entire genome of nonhuman adenovirus (porcine adenovirus type 3, PAd3 or bovine adenovirus type 3, BAd3) with deletions in E1 and E3 regions with or without insertion in E1 were generated by homologous recombination in E. coli BJ5183. The HA gene of H5N1, flanked by the CMV promoter and the bovine growth hormone BGH polyadenylation signal was cloned into pDS2 (Bangari & Mittal, Virus Research 105:127-136, 2004) at the AvrII site to obtain pDS2-H5. Using homologous recombination in E. coli BJ5183 as described in van Olphen & Mittal, J. Virol. Methods 77:125-129, 1999, with respect to bovine adenovirus, pPAd-H5 (a genomic plasmid with an avian HA insertion into the E1A gene region of porcine adenovirus) was generated by cotransformation of E. coli with E3-deleted PAd3 genomic DNA and StuI linearized pDS2-H5.

To generate HA of H5N1 influenza from the PAd3 vector, monolayer cultures of FPRT HE1-5 cells (an E1 expressing porcine cell line described in Bangari & Mittal, Virus Res. 105:127-136, 2004) were transfected with PacI-digested pPAd-H5 (5 μg/60-mm dish) using LIPOFECTIN®-mediated transfection according to the manufacturer's recommendations. Recombinant virus-induced cytopathic effect was visible in 2-3 weeks post-transfection.

Replication-defective recombinant PAd3 vector (PAd-H5HA) containing the full-length coding region of the HA gene of H5N1 virus (HK/156/97) inserted in the early region 1 (E1) of PAd3 genome was expressed efficiently in FPRT HE1-5 cells as demonstrated by western blotting. A PAd with deletions of E1 and E3 regions (PAd-ΔE1E3) served as a negative control.

Similarly, a replication-defective recombinant BAd3 vector (BAd-H5HA) including the full-length coding region of the HA gene of H5N1 virus (HK/156/97) inserted in the early region 1 (E1) of BAd3 genome was expressed efficiently in FBRT-HE1 cells that express BAd3 E1 (van Olphen et al., Virology 295:108-118, 2002). A BAd3 with deletions of E1 and E3 regions (BAd-ΔE1E3) served as a negative control.

### Example 17

### Inhibition of an Inflammatory Response by the C-Terminal Domain of RIG-I

This example describes the ability of vaccines containing C-terminal RIG-I to suppress the expression of inflammatory cytokines post influenza infection.

To assess if vaccines containing C-terminal RIG-I suppress the inflammatory response after the animals are infected, young Balb/c mice are infected with either A/Indonesia/5/05 or antigenically distinct H5N1 viruses. The vaccine vaccines containing C-terminal RIG-I is administered once on different days post-infection (day 0, 1, 2, 3, 4, 5, 6, 7, or 8) to groups of mice. The levels of inflammatory cytokines such as interleukin-6, tumor necrosis factor-α and interferon-α are determined.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A method for inhibiting a viral infection in a subject, comprising:
selecting a subject in whom the viral infection is to be inhibited; and
administering to the subject an effective amount of a recombinant adenovirus vector comprising a nucleic acid sequence encoding at least one caspase recruitment domain (CARD) from MDA5 or RIG-I, wherein the recombinant adenovirus vector does not comprise a nucleic acid sequence encoding a MDA5 or RIG-I helicase domain, thereby inhibiting the viral infection in the subject.

2. The method of claim 1, wherein the nucleic acid sequence encodes the at least one CARD from RIG-I and comprises a nucleic acid sequence encoding an amino acid sequence at least 95% identical to amino acids 1-87 of the amino acid sequence set forth as SEQ ID NO:1, a nucleic acid sequence encoding an amino acid sequence at least 95% identical to amino acids 92-172 of the amino acid sequence set forth as SEQ ID NO:1, or a nucleic acid sequence encoding an amino acid sequence at least 95% identical to amino acids 1-284 of the amino acid sequence set forth as SEQ ID NO:1 and wherein the nucleic acid sequence encoding at least one CARD from MDA5 comprises a nucleic acid sequence encoding an amino acid sequence at least 95% identical to amino acids 7-97 of the amino acid sequence set forth as SEQ ID NO:3, a nucleic acid sequence encoding an amino acid sequence at least 95% identical to amino acids 110-190 of the amino acid sequence set forth as SEQ ID NO:3, or a nucleic acid sequence encoding an amino acid sequence at least 95% identical to amino acids 1-196 of the amino acid sequence set forth as SEQ ID NO:3.

3.-7. (canceled)

8. The method of claim 1, wherein the viral infection is a RNA viral infection.

9. The method of claim 8, wherein the viral infection is an influenza infection.

10. The method of claim 9, wherein the influenza infection is an influenza A infection.

11. The method of claim 1, wherein the recombinant adenovirus vector is a human adenovirus vector.

12. The method of claim 1, wherein the recombinant adenovirus vector is a non-human adenovirus vector.

13. The method of claim 12, wherein the non-human adenovirus vector is a porcine adenovirus vector, a bovine adenovirus vector, a canine adenovirus vector, a murine adenovirus vector, an ovine adenovirus vector, an avian adenovirus vector or a simian adenovirus vector.

14. The method of claim 1, wherein the recombinant adenovirus vector is a replication defective adenovirus vector.

15. The method of claim 14, wherein the replication defective adenovirus vector comprises a mutation in at least one of an E1 region gene or an E3 region gene.

16. The method of claim 1, wherein the recombinant adenovirus vector further comprises a nucleic acid sequence encoding at least one viral antigen.

17. The method of claim 16, wherein the at least one viral antigen comprises at least one of an internal protein, an external protein, or a combination thereof.

18. The method of claim 17, wherein the at least one viral antigen comprises at least one influenza antigen.

19. The method of claim 17, wherein the at least one influenza antigen comprises at least one of an influenza hemagglutinin (HA) antigen or an influenza neuraminidase (NA) antigen.

20. The method of claim 18, wherein the at least one influenza antigen comprises an H5N1 strain antigen, an H7N7 strain antigen, or an H9N2 strain antigen.

21. The method of claim 18, further comprising a nucleic acid sequence that encodes at least one influenza internal protein.

22. The method of claim 21, wherein the influenza internal protein is an M1 protein, an M2 protein, an NP protein, a PB1 protein, a PB2 protein, an NS1 protein, an NS2 protein, or a combination thereof.

23. The method of claim 21, wherein the internal protein is of an H1N1, H2N2 or H3N2 influenza strain.

24. The method of claim 1, wherein selecting the subject comprises selecting a subject who already has a viral infection.

25. The method of claim 1, wherein selecting the subject comprises selecting a subject in whom an immunogenic response to an antigen is to be enhanced.

26. The method of claim 25, further comprising administering a viral vaccine to the subject, and wherein inhibiting the viral infection comprises enhancing the effectiveness of the viral vaccine.

27. The method of claim 26, wherein the vaccine is an influenza vaccine.

28. The method of claim 26, wherein the influenza vaccine is a vaccine against one or more avian or pandemic strains of influenza.

29. The method of claim 26, wherein the one or more avian or pandemic strains of influenza comprise influenza strain H5N1, strain H7N7, strain H9N2, or a combination thereof.

30. The method of claim 26, wherein the recombinant adenovirus vector is administered prior to administering a viral vaccine, concurrent with administering viral vaccine, or administered after administering a viral vaccine.

31. The method of claim 26, wherein the viral vaccine comprises a second adenovirus vector comprising a nucleic acid sequence that encodes at least one viral antigen.

32. The method of claim 31, wherein the at least one viral antigen comprises at least one of an internal protein, an external protein, or a combination thereof.

33. The method of claim 31, wherein the at least one viral antigen comprises at least one RNA virus antigen.

34. The method of claim 33, wherein the at least one virus antigen comprises at least one influenza antigen.

35. The method of claim 34, wherein the at least one influenza antigen comprises at least one of an influenza HA antigen or an influenza NA antigen.

36. The method of claim 34, wherein the at least one influenza antigen comprises an H5N1 strain antigen, an H7N7 strain antigen, or an H9N2 strain antigen.

37. The method of claim 34, wherein the at least one influenza antigen comprises at least one influenza internal protein.

38. The method of claim 37, wherein the influenza internal protein is an M1 protein, an M2 protein, an NP protein, a PB1 protein, a PB2 protein, an NS1 protein, and NS2 protein, or a combination thereof.

39. The method of claim 38, wherein the internal protein is of an H1N1, H2N2, or H3N2 influenza strain.

40. The method of claim 31, wherein the second adenovirus vector is a replication defective adenovirus vector.

41. The method of claim 40, wherein the replication defective adenovirus comprises a mutation in at least one of an E1 region gene and an E3 region gene.

42. The method of claim 31, wherein the second adenovirus vector is a human adenovirus vector.

43. The method of claim 31, wherein the second adenovirus vector is a non-human adenovirus vector.

44. The method of claim 43, wherein the non-human adenovirus vector is a porcine adenovirus vector, a bovine adenovirus vector, a canine adenovirus vector, a murine adenovirus vector, an ovine adenovirus vector, an avian adenovirus vector or a simian adenovirus vector.

45. The method of claim 1, further comprising administering to the subject an effective amount of Flt3 ligand or a nucleic acid that encodes Flt3 ligand, wherein the Flt3 ligand increases the number of dendritic cells in the subject.

46. A recombinant adenovirus vector comprising a nucleic acid sequence encoding at least one caspase recruitment domain (CARD) from MDA5 or RIG-I or RIG-I, wherein the recombinant adenovirus vector does not comprise a nucleic acid sequence encoding a helicase domain.

47. The recombinant adenovirus vector of claim 46, wherein the nucleic acid sequence encoding at least one CARD from RIG-I comprises a nucleic acid sequence encoding an amino acid sequence at least 95% identical to amino acids 1-87 of the amino acid sequence set forth as SEQ ID NO:1, a nucleic acid sequence encoding an amino acid sequence at least 95% identical to amino acids 92-172 of the amino acid sequence set forth as SEQ ID NO:1, or a nucleic acid sequence encoding an amino acid sequence at least 95% identical to amino acids 1-284 of the amino acid sequence set forth as SEQ ID NO:1 and wherein the nucleic acid sequence encoding at least one CARD from MDA5 comprises a nucleic acid sequence encoding an amino acid sequence at least 95% identical to amino acids 7-97 of the amino acid sequence set forth as SEQ ID NO:3, a nucleic acid sequence encoding an amino acid sequence at least 95% identical to amino acids 110-190 of the amino acid sequence set forth as SEQ ID NO:3, or a nucleic acid sequence encoding an amino acid sequence at least 95% identical to amino acids 1-196 of the amino acid sequence set forth as SEQ ID NO:3.

48.-52. (canceled)

53. The recombinant adenovirus vector of claim 46, further comprising a nucleic acid sequence that encodes Flt3 ligand.

54. The recombinant adenovirus vector of claim 46, further comprising a nucleic acid sequence that encodes at least one viral antigen.

55. The recombinant adenovirus vector of claim 54, wherein the at least one viral antigen comprises at least one of an internal protein, an external protein, or a combination thereof.

56. The recombinant adenovirus vector of claim 54, wherein the at least one viral antigen comprises at least one RNA virus antigen.

57. The recombinant adenovirus vector of claim 56, wherein the at least one RNA viral antigen comprises at least one influenza antigen.

58. The recombinant adenovirus vector of claim 57, wherein the at least one influenza antigen comprises at least one of an influenza HA antigen or an influenza NA antigen.

59. The recombinant adenovirus vector of claim 57, wherein the influenza antigen comprises an H5N1 strain antigen, an H7N7 strain antigen, or an H9N2 strain antigen.

60. The recombinant adenovirus vector of claim 46, further comprising a nucleic acid sequence that encodes at least one influenza internal protein.

61. The recombinant adenovirus vector of claim 60, wherein the influenza internal protein is an M1 protein, an M2 protein, an NP protein, a PB1 protein, a PB2 protein, an NS1 protein, an NS2 protein, or a combination thereof.

62. The recombinant adenovirus vector of claim 61, wherein the internal protein is of an H1N1, H2N2 or H3N2 influenza strain.

63. The recombinant adenovirus vector of claim 46, wherein the adenovirus vector is a human adenovirus vector.

64. The recombinant adenovirus vector of claim 46, wherein the adenovirus vector is a non-human adenovirus vector.

65. The recombinant adenovirus vector of claim 64, wherein the non-human adenovirus vector is a porcine adenovirus vector, a bovine adenovirus vector, a canine adenovirus vector, a murine adenovirus vector, an ovine adenovirus vector, an avian adenovirus vector or a simian adenovirus vector.

66. The recombinant adenovirus vector of claim 46, wherein the adenovirus vector is a replication defective adenovirus vector.

67. The recombinant adenovirus vector of claim 66, wherein the replication defective adenovirus comprises a mutation in at least one of an E1 region gene and an E3 region gene.

68. A composition comprising the recombinant adenovirus vector of claim 46 and a pharmaceutically acceptable carrier.

69. A method of inhibiting viral replication in a cell comprising contacting the cell with the adenoviral vector of claim 46, thereby inhibiting viral replication in the cell.


SEQUENCE LISTING (6 sequences)

SEQ ID NO: 1 - protein, 925 aa, Homo sapiens
MTTEQRRSLQAFQDYIRKTLDPTYILSYMAPWFREEEVQYIQAEKNNKGPMEAATLFLKFLLELQEEGWFRGFLDALDHAGYSGLYEAIESWDFKKIEKLEEYRLLLKRLQPEFKTRIIPTDIISDLSECLINQECEEILQICSTKGMMAGAEKLVECLLRSDKENWPKTLKLALEKERNKFSELWIVEKGIKDVETEDLEDKMETSDIQIFYQEDPECQNLSENSCPPSEVSDTNLYSPFKPRNYQLELALPAMKGKNTIICAPTGCGKTFVSLLICEHHLKKFPQGQKGKVVFFANQIPVYEQQKSVFSKYFERHGYRVTGISGATAENVPVEQIVENNDIIILTPQILVNNLKKGTIPSLSIFTLMIFDECHNTSKQHPYNMIMFNYLDQKLGGSSGPLPQVIGLTASVGVGDAKNTDEALDYICKLCASLDASVIATVKHNLEELEQVVYKPQKFFRKVESRISDKFKYIIAQLMRDTESLAKRICKDLENLSQIQNREFGTQKYEQWIVTVQKACMVFQMPDKDEESRICKALFLYTSHLRKYNDALIISEHARMKDALDYLKDFFSNVRAAGFDEIEQDLTQRFEEKLQELESVSRDPSNENPKLEDLCFILQEEYHLNPETITILFVKTRALVDALKNWIEGNPKLSFLKPGILTGRGKTNQNTGMTLPAQKCILDAFKASGDHNILIATSVADEGIDIAQCNLVILYEYVGNVIKMIQTRGRGRARGSKCFLLTSNAGVIEKEQINMYKEKMMNDSILRLQTWDEAVFREKILHIQTHEKFIRDSQEKPKPVPDKENKKLLCRKCKALACYTADVRVIEECHYTVLGDAFKECFVSRPHPKPKQFSSFEKRAKIFCARQNCSHDWGIHVKYKTFEIPVIKIESFVVEDIATGVQTLYSKWKDFHFEKIPFDPAEMSK

SEQ ID NO: 2 - DNA, 2778 bp, Homo sapiens
atgaccaccgagcagcgacgcagcctgcaagccttccaggattatatccggaagaccctggaccctacctacatcctgagctacatggccccctggtttagggaggaagaggtgcagtatattcaggctgagaaaaacaacaagggcccaatggaggctgccacactttttctcaagttcctgttggagctccaggaggaaggctggttccgtggctttttggatgccctagaccatgcaggttattctggactttatgaagccattgaaagttgggatttcaaaaaaattgaaaagttggaggagtatagattacttttaaaacgtttacaaccagaatttaaaaccagaattatcccaaccgatatcatttctgatctgtctgaatgtttaattaatcaggaatgtgaagaaattctacagatttgctctactaaggggatgatggcaggtgcagagaaattggtggaatgccttctcagatcagacaaggaaaactggcccaaaactttgaaacttgctttggagaaagaaaggaacaagttcagtgaactgtggattgtagagaaaggtataaaagatgttgaaacagaagatcttgaggataagatggaaacttctgacatacagattttctaccaagaagatccagaatgccagaatcttagtgagaattcatgtccaccttcagaagtgtctgatacaaacttgtacagcccatttaaaccaagaaattaccaattagagcttgctttgcctgctatgaaaggaaaaaacacaataatatgtgctcctacaggttgtggaaaaacctttgtttcactgcttatatgtgaacatcatcttaaaaaattcccacaaggacaaaaggggaaagttgtcttttttgcgaatcagatcccagtgtatgaacagcagaaatctgtattctcaaaatactttgaaagacatgggtatagagttacaggcatttctggagcaacagctgagaatgtcccagtggaacagattgttgagaacaatgacatcatcattttaactccacagattcttgtgaacaaccttaaaaagggaacgattccatcactatccatctttactttgatgatatttgatgaatgccacaacactagtaaacaacacccgtacaatatgatcatgtttaattatctagatcagaaacttggaggatcttcaggcccactgccccaggtcattgggctgactgcctcggttggtgttggggatgccaaaaacacagatgaagccttggattatatctgcaagctgtgtgcttctcttgatgcgtcagtgatagcaacagtcaaacacaatctggaggaactggagcaagttgtttataagccccagaagtttttcaggaaagtggaatcacggattagcgacaaatttaaatacatcatagctcagctgatgagggacacagagagtctggcaaagagaatctgcaaagacctcgaaaacttatctcaaattcaaaatagggaatttggaacacagaaatatgaacaatggattgttacagttcagaaagcatgcatggtgttccagatgccagacaaagatgaagagagcaggatttgtaaagccctgtttttatacacttcacatttgcggaaatataatgatgccctcattatcagtgagcatgcacgaatgaaagatgctctggattacttgaaagacttcttcagcaatgtccgagcagcaggattcgatgagattgagcaagatcttactcagagatttgaagaaaagctgcaggaactagaaagtgtttccagggatcccagcaatgagaatcctaaacttgaagacctctgcttcatcttacaagaagagtaccacttaaacccagagacaataacaattctctttgtgaaaaccagagcacttgtggacgctttaaaaaattggattgaaggaaatcctaaactcagttttctaaaacctggcatattgactggacgtggcaaaacaaatcagaacacaggaatgaccctcccggcacagaagtgtatattggatgcattcaaagccagtggagatcacaatattctgattgccacctcagttgctgatgaaggcattgacattgcacagtgcaatcttgtcatcctttatgagtatgtgggcaatgtcatcaaaatgatccaaaccagaggcagaggaagagcaagaggtagcaagtgcttccttctgactagtaatgctggtgtaattgaaaaagaacaaataaacatgtacaaagaaaaaatgatgaatgactctattttacgccttcagacatgggacgaagcagtatttagggaaaagattctgcatatacagactcatgaaaaattcatcagagatagtcaagaaaaaccaaaacctgtacctgataaggaaaataaaaaactgctctgcagaaagtgcaaagccttggcatgttacacagctgacgtaagagtgatagaggaatgccattacactgtgcttggagatgcttttaaggaatgctttgtgagtagaccacatcccaagccaaagcagttttcaagttttgaaaaaagagcaaagatattctgtgcccgacagaactgcagccatgactggggaatccatgtgaagtacaagacatttgagattccagttataaaaattgaaagttttgtggtggaggatattgcaactggagttcagacactgtactcgaagtggaaggactttcattttgagaagataccatttgatccagcagaaatgtccaaatga

SEQ ID NO: 3 - protein, 1025 aa, Homo sapiens [sequence omitted]

SEQ ID NO: 4 - DNA, 3078 bp, Homo sapiens
atgtcgaatgggtattccacagacgagaatttccgctatctcatctcgtgcttcagggccagggtgaaaatgtacatccaggtggagcctgtgctggactacctgacctttctgcctgcagaggtgaaggagcagattcagaggacagtcgccacctccgggaacatgcaggcagttgaactgctgctgagcaccttggagaagggagtctggcaccttggttggactcgggaattcgtggaggccctccggagaaccggcagccctctggccgcccgctacatgaaccctgagctcacggacttgccctctccatcgtttgagaacgctcatgatgaatatctccaactgctgaacctccttcagcccactctggtggacaagcttctagttagagacgtcttggataagtgcatggaggaggaactgttgacaattgaagacagaaaccggattgctgctgcagaaaacaatggaaatgaatcaggtgtaagagagctactaaaaaggattgtgcagaaagaaaactggttctctgcatttctgaatgttcttcgtcaaacaggaaacaatgaacttgtccaagagttaacaggctctgattgctcagaaagcaatgcagagattgagaatttatcacaagttgatggtcctcaagtggaagagcaacttctttcaaccacagttcagccaaatctggagaaggaggtctggggcatggagaataactcatcagaatcatcttttgcagattcttctgtagtttcagaatcagacacaagtttggcagaaggaagtgtcagctgcttagatgaaagtcttggacataacagcaacatgggcagtgattcaggcaccatgggaagtgattcagatgaagagaatgtggcagcaagagcatccccggagccagaactccagctcaggccttaccaaatggaagttgcccagccagccttggaagggaagaatatcatcatctgcctccctacagggagtggaaaaaccagagtggctgtttacattgccaaggatcacttagacaagaagaaaaaagcatctgagcctggaaaagttatagttcttgtcaataaggtactgctagttgaacagctcttccgcaaggagttccaaccatttttgaagaaatggtatcgtgttattggattaagtggtgatacccaactgaaaatatcatttccagaagttgtcaagtcctgtgatattattatcagtacagctcaaatccttgaaaactccctcttaaacttggaaaatggagaagatgctggtgttcaattgtcagacttttccctcattatcattgatgaatgtcatcacaccaacaaagaagcagtgtataataacatcatgaggcattatttgatgcagaagttgaaaaacaatagactcaagaaagaaaacaaaccagtgattccccttcctcagatactgggactaacagcttcacctggtgttggaggggccacgaagcaagccaaagctgaagaacacattttaaaactatgtgccaatcttgatgcatttactattaaaactgttaaagaaaaccttgatcaactgaaaaaccaaatacaggagccatgcaagaagtttgccattgcagatgcaaccagagaagatccatttaaagagaaacttctagaaataatgacaaggattcaaacttattgtcaaatgagtccaatgtcagattttggaactcaaccctatgaacaatgggccattcaaatggaaaaaaaagctgcaaaaaaaggaaatcgcaaagaacgtgtttgtgcagaacatttgaggaagtacaatgaggccctacaaattaatgacacaattcgaatgatagatgcgtatactcatcttgaaactttctataatgaagagaaagataagaagtttgcagtcatagaagatgatagtgatgagggtggtgatgatgagtattgtgatggtgatgaagatgaggatgatttaaagaaacctttgaaactggatgaaacagatagatttctcatgactttattttttgaaaacaataaaatgttgaaaaggctggctgaaaacccagaatatgaaaatgaaaagctgaccaaattaagaaataccataatggagcaatatactaggactgaggaatcagcacgaggaataatctttacaaaaacacgacagagtgcatatgcgctttcccagtggattactgaaaatgaaaaatttgctgaagtaggagtcaaagcccaccatctgattggagctggacacagcagtgagttcaaacccatgacacagaatgaacaaaaagaagtcattagtaaatttcgcactggaaaaatcaatctgcttatcgctaccacagtggcagaagaaggtctggatattaaagaatgtaacattgttatccgttatggtctcgtcaccaatgaaatagccatggtccaggcccgtggtcgagccagagctgatgagagcacctacgtcctggttgctcacagtggttcaggagttatcgaacatgagacagttaatgatttccgagagaagatgatgtataaagctatacattgtgttcaaaatatgaaaccagaggagtatgctcataagattttggaattacagatgcaaagtataatggaaaagaaaatgaaaaccaagagaaatattgccaagcattacaagaataacccatcactaataactttcctttgcaaaaactgcagtgtgctagcctgttctggggaagatatccatgtaattgagaaaatgcatcacgtcaatatgaccccagaattcaaggaactttacattgtaagagaaaacaaagcactgcaaaagaagtgtgccgactatcaaataaatggtgaaatcatctgcaaatgtggccaggcttggggaacaatgatggtgcacaaaggcttagatttgccttgtctcaaaataaggaattttgtagtggttttcaaaaataattcaacaaagaaacaatacaaaaagtgggtagaattacctatcacatttcccaatcttgactattcagaatgctgtttatttagtgatgaggattag

SEQ ID NO: 5 - protein, 9 aa, Influenza A virus
IYSTVASSL

SEQ ID NO: 6 - protein, 9 aa, Influenza A virus
TYQRTRALV